SEQUENCE LISTING (10 sequences)

SEQ ID NO: 1 - DNA, 30 bp, Artificial
  Oligonucleotides for immobilization
ttttttttttaatgatacggcgaccaccga

SEQ ID NO: 2 - DNA, 31 bp, Artificial
  Oligonucleotides for immobilization
ttttttttttcaagcagaagacggcatacga

SEQ ID NO: 3 - DNA, 32 bp, Artificial
  Hybridization probe
agagtgtagatctcggtggtcgccgtatcatt

SEQ ID NO: 4 - DNA, 52 bp, Artificial
  fluorescent oligonucleotide
agagtgtagatctcggtggtcgccgtatcattcagcatgcactttttttttt

SEQ ID NO: 5 - DNA, 16 bp, Artificial
  Sph I restriction site
aaagtgcatgctgaat

SEQ ID NO: 6 - DNA, 25 bp, Artificial
  Oligo (dT)25
ttttttttttttttttttttttttt

SEQ ID NO: 7 - DNA, 25 bp, Artificial
  Olido (dA) 25
aaaaaaaaaaaaaaaaaaaaaaaaa

SEQ ID NO: 8 - DNA, 340 bp, Artificial
  G3PDH probe
tcggtggtcgccgtatcatttgtgagggagatgctcagtgttgggggccgagttgggatagggcctctcttgctcagtgtccttgctggggtgggtggtccagggtttcttactccttggaggccatgtaggccatgaggtccaccaccctgttgctgtagccgtattcattgtcataccaggaaatgagcttgacaaagttgtcattgagagcaatgccagccccggcatcgaaggtggaagagtgggagttgctgttgaagtcgcaggagacaacctggtcctcagtgtagcccaagatgcccttcagtgggccctcagatgcctgcttcaccacctt

SEQ ID NO: 9 - DNA, 340 bp, Artificial
  G3PDH probe
tcggtggtcgccgtatcattcttgatgtcatcatacttggcaggtttctccaggcggcacgtcagatccacgacggacacattgggggtaggaacacggaaggccatgccagtgagcttcccgttcagctctgggatgaccttgcccacagccttggcagcaccagtggatgcagggatgatgttctgggcagccccacggccatcacgccacagctttccagaggggccatccacagtcttctgggtggcagtgatggcatggactgtggtcatgagcccttccacaatgccaaagttgtcatggatgaccttggccaggggggctaagcagttggtgg

SEQ ID NO: 10 - DNA, 345 bp, Artificial
  G3PDH probe
tcggtggtcgccgtatcatttgcaggatgcattgctgacaatcttgagtgagttgtcatatttctcgtggttcacacccatcacaaacatgggggcatcggcagaaggggcggagatgatgacccttttggctccacccttcaagtgggccccggccttctccatggtggtgaagacaccagtagactccacgacatactcagcaccggcctcaccccatttgatgttagtggggtctcgctcctggaagatggtgatgggcttcccgttgatgacaagcttcccattctcggccttgactgtgccgttgaatttgccgtgagtggagtcatactggaacatgta

(19) United States
(12) Patent Application Publication
(10) Pub. No.: US 2015/0141269 A1
(43) Pub. Date: May 21, 2015

(54) HYBRIDIZATION-BASED REPLICATION OF NUCLEIC ACID MOLECULES

(71) Applicant: MAX-PLANCK-GESELLSCHAFT ZUR FÖRDERUNG DER WISSENSCHAFTEN E.V., Munich (DE)

(72) Inventors: Aleksey Soldatov, Berlin (DE); Tatiana Borodina, Berlin (DE); Hans Lehrach, Berlin (DE)

(73) Assignee: MAX-PLANCK-GESELLSCHAFT ZUR FÖRDERUNG DER WISSENSCHAFTEN E.V., Munich (DE)

(21) Appl. No.: 14/388,669

(22) PCT Filed: Apr. 3, 2013
(86) PCT No.: PCT/EP2013/057052

§ 371 Date: Sep. 26, 2014

Related U.S. Application Data
(60) Provisional application No. 61/687,681, filed on Apr. 30, 2012.

(30) Foreign Application Priority Data
Apr. 3, 2012 (EP) 12163055.2

(51) Int. Cl. C12N 15/10; C12Q 1/68

(52) U.S. Cl. CPC C12N 15/1093; C12Q 1/6874
USPC 506/3; 506/32

(57) ABSTRACT

The present invention provides methods for replication of nucleic acid molecules distributed on a surface or within a layer by transferring them to a target surface covered with oligonucleotides, and fixation of transferred molecules by hybridization to complementary sequences.

In the present invention a replica of nucleic acid molecules distributed on a surface or within a layer is created by transferring them to a target surface covered with oligonucleotides, and fixation of transferred molecules by hybridization to complementary sequences. Oligonucleotides on the target surface may be complementary to the replicated nucleic acid molecules or hybridization may occur through adapter oligonucleotides complementary both to the oligonucleotides on the target surface and to the replicated nucleic acid molecules. Replicated molecules can be sequenced directly on the target surface. Positions of sequenced molecules allow determination of a spatial distribution of nucleic acid molecules in the original sample.

### BACKGROUND OF THE INVENTION

Biological processes are spatially organized. They rely upon the interplay of many different components forming an intricate structure of cells, tissues and organisms. Molecules participating in these processes have a certain spatial distribution. Understanding the biological processes is critically dependent on a detailed knowledge of this distribution.

Objects with two-dimensional distribution of nucleic acid molecules, for example tissue sections, are widely studied. There exist methods for nucleic acid analysis in tissue sections, for example in situ hybridization or in situ PCR. However, not all molecular biology methods are applicable when working with tissue sections.

Two-dimensional tissue sections are convenient objects to study distribution of molecules. Several sequential sections restore a 3D spatial location of molecules. However, many molecular biology methods, for example sequencing, cannot be performed directly in tissue sections. It would be advantageous to be able to transfer molecules from the tissue section to another surface or into solution, where appropriate methods of analysis could be performed. However, such transfer raises questions of keeping information about initial distribution of the nucleic acid molecules.

Replication of 2D distributed objects (nucleic acid molecules, cells) has been long used in molecular biology. Main purposes are to perform analysis which is not possible with original sample and (ii) multiplying 2D sample for several analyses.

Southern and Northern methods are known, wherein nucleic acid molecules are transferred from gel to membrane. Membrane allows analyzing transferred molecules by hybridization preserving the relative distribution they had in gel. Replica of DNA of library clones on membranes is used to search for particular clones using hybridization. Replication of bacterial colonies to other plates allows analyzing in parallel, for example, their resistance to several antibiotics.

In the last decade, several methods were suggested for multiplying nucleic acid arrays by replication. In one approach nucleic acid array features are first amplified on the array, then the array with amplified features is brought into tight contact with transfer support, to which parts of amplified molecules are transferred and get covalently attached (U.S. Pat. No. 7,785,790). In the nanostamping approach nucleic acid molecules hybridised to sample surface are brought into direct contact with capturing groups on the target surface. Chemical binding with the target surface is stronger than hybridization and after separating surfaces, nucleic acid molecules remain on the target surface (U.S. Pat. No. 7,862,849).

The general principle of replication is bringing into contact a surface with 2D distributed nucleic acid molecules with a target surface, to which they are transferred by diffusion or direct contact. So far nucleic acid molecules have been transferred to surfaces were they were captured either physically (stuck in gel) or by chemical bonds (covalent, ion exchange, affinity) involving certain reactive groups on the nucleic acid molecules and on the target surface, but not involving the nucleotide sequence of the molecules.

Objective of the present invention is to provide a method capable of preserving the information about spatial distribution of nucleic acid molecules transferred from a surface to another surface.

This objective is solved by the present methods as shown below. Further preferred embodiments of the present invention are disclosed in the dependent claims, the description, the figures and the examples.

Surprisingly it was found that methods according to the present invention allow the transfer of nucleic acid molecules to another surface preserving information about their original positions.

### DESCRIPTION OF THE INVENTION

In the present invention replica of nucleic acid molecules distributed on a surface or within a layer is created by transferring them to a target surface covered with oligonucleotides, and fixation of transferred molecules by hybridization to complementary sequences. Oligonucleotides on the target surface may be complementary to the replicated nucleic acid molecules (FIG. 1A) or hybridization may occur through adapter oligonucleotides complementary both to the oligonucleotides on the target surface and to the replicated nucleic acid molecules (FIG. 1B).

Hybridization has several advantages for fixation of transferred molecules on the target surface:
i. strong and reliable binding (if double-stranded region is long enough);
ii. high specificity;
iii. binding is easily reversible;
iv. does not require any modifications of nucleic acids;
v. may be used for organization of such enzymatic reactions as primer extension, ligation, on surface amplification (RCA, bridge amplification, etc.).

All these advantages make hybridization-based replicas a convenient instrument for the analysis of two-dimensionally (2D) distributed nucleic acid molecules. Strong and reliable binding allows using advantages of surface-immobilized reactions: easy substitution and removal of reaction components, high yield and selectivity. High specificity allows selecting desired components from complex nucleic acid mixtures, for example polyadenylated RNA or particular genomic regions (transcripts) from tissue sections. Reversible binding allows easily switch from the surface-based reaction to the reaction in solution. No need in modification is very helpful for complex enzymatic procedures. Hybridized molecules may be a convenient substrate for such enzymatic reactions as primer extension or ligation (FIG. 3). Hybridization-based replicas may provide templates for surface amplification reactions: RCA (FIG. 4) or bridge amplification.

The present invention is directed to methods for preserving information about original spatial distribution of nucleic acid molecules transferred from a surface to another surface or into solution. The present invention is further directed to method of transfer of nucleic acids molecules located on an original surface or within a layer to another surface preserving relative spatial distribution of nucleic acid molecules resembling the original distribution.

This is accomplished by the use of hybridization of nucleic acid molecules for with complementary nucleic acid molecules located on the target surface to fix transferred molecules in particular positions (FIG. 1) creating replicas of nucleic acids molecules located either on a surface or within a layer, wherein said creating replicas is obtaining on a target surface the relative distribution of nucleic acid molecules resembling the original distribution.

Hybridization-based replicas are stable, because hybridization keeps the transferred nucleic acid molecules in fixed positions on the target surface. Capturing of nucleic acid molecules based on hybridization makes the replication method highly selective, since only nucleic acid molecules having complementary sequences will be hold on the target surface. Specificity and speed of hybridization may be controlled by temperature and composition of hybridization solution.

The method of the invention does not require direct contact of original surface bearing nucleic acid molecules with the target surface. This means that
(i) the transfer may be performed between large solid surfaces, which can't form uniform tight contact and
(ii) the method may be applied to transfer nucleic acid molecules not only from a surface, but also from a layer with distributed nucleic acid molecules.

One preferred method of the invention refers to a method of transfer of nucleic acid molecules to a target surface, preserving their relative spatial distribution resembling the original distribution, wherein said nucleic acids molecules are fixed on a said target surface by hybridization to complementary sequences, comprising the following steps:

a) providing a sample with nucleic acid molecules located either on a surface or within a layer;
b) providing a target surface with immobilized oligonucleotides;
c) assembling sample with nucleic acid molecules from (a) against the target surface from (b) in such a way, that a distance from positions of said nucleic acids to the target surface is smaller than the distortion acceptable for the replica and with solution in between sample and target surface;
d) providing conditions for diffusion of nucleic acid molecules from the sample to the target surface and hybridization-based binding of nucleic acid molecules from the sample to the oligonucleotides on the target surface.

Before assembling the sample with nucleic acid molecules from (a) against the target surface from (b) conditions to minimize shift of molecules from the original positions may be provided if nucleic acid molecules are not attached to the sample for instance by cooling the sample or the use of agents which minimize shift. If the nucleic acid molecules are attached to the sample, provision of conditions for gradual releasing of nucleic acid molecules is part of the inventive method. This can be done before or after assembling.

The term “replica” as used herein refers to a copy of the distribution of nucleic acid molecules with preservation of their original distribution to a target surface by hybridization. The target surface with the transferred nucleic acid molecules held by hybridization with preservation of their original distribution is the created replica. Thus, replica is obtaining on a target surface the relative distribution of nucleic acid molecules resembling the original distribution.

An “oligonucleotide” as used herein is a short, single stranded nucleic acid polymer (DNA, RNA, LNA, PNA or mixture of those), typically with one hundred fifty or fewer bases of a known sequence. Although for the purposes of the present invention, the oligonucleotides can have more or less bases and have preferably between 20 and 60 bases. Oligonucleotides can commonly be made in the laboratory with any user-specified sequence by solid-phase chemical synthesis. The term “type of oligonucleotide” as used herein means an oligonucleotide of a specific sequence, which can be present in several copies. The term “nucleic acids” or “nucleic acid molecule” comprises single- and double-stranded RNA, DNA, oligonucleotides or hybridization products of those.

The replication method according to the invention preferably comprises the following steps:

a) providing sample with nucleic acid molecules located either on a surface or within a layer;
b) providing target surface with nucleic acid molecules, capable to hybridization-based binding to nucleic acid molecules from (a);
c) (option 1) if nucleic acid molecules are not attached to the sample, providing conditions to minimize shift of molecules from the original positions;
c′) (option 2) if nucleic acid molecules are attached to the sample, providing conditions for gradual releasing of nucleic acid molecules;
d) assembling sample with nucleic acid molecules from (a) against the target surface from (b) with solution in between, such that nucleic acid molecules from the sample can reach target surface by diffusion through solution;
c″) (option 3) if nucleic acid molecules are attached to the sample, providing conditions for releasing nucleic acid molecules from the original positions in the sample,
d) providing conditions for diffusion of nucleic acid molecules from the sample to the target surface and hybridization-based binding of nucleic acid molecules from the sample to the nucleic acid molecules on the target surface;
e) (optional) providing conditions for slowing down the formation of new hybrids of nucleic acid molecules ;
f) disassembling the sample (a) and target surface (b).

Another preferred method of transfer of nucleic acid molecules to a target surface, preserving their relative spatial distribution resembling the original distribution, wherein said nucleic acids molecules are fixed on said target surface by hybridization, comprising the following steps:

a) providing the sample containing nucleic acid molecules located either on the surface of the sample or within the sample;
b) providing a target surface with immobilized oligonucleotides;
c) if the nucleic acid molecules are not attached to the sample, providing conditions to minimize shift of the nucleic acid molecules from the original positions on or within the sample; or
c′) if nucleic acid molecules are attached to the sample, providing conditions for releasing the nucleic acid molecules;
d) assembling the sample and the target surface with a medium in between sample and target surface;
e) providing conditions for diffusion of the nucleic acid molecules from the sample to the target surface and hybridization-based binding of nucleic acid molecules to the oligonucleotides on the target surface.

Optionally in said method step c′), namely releasing of nucleic acid molecules is performed after step d) (assembling of sample and target surface) only.

One preferred method according to the invention comprises the following steps:

if the nucleic acid molecules are not attached to the sample:
a) providing the sample containing nucleic acid molecules located either on the surface or within the sample;
b) providing a target surface with immobilized oligonucleotides;
c) providing conditions to minimize shift of the nucleic acid molecules from the original positions on or within the sample;
d) assembling the sample and the target surface with a medium in between sample and target surface;
e) providing conditions for diffusion of the nucleic acid molecules from the sample to the target surface and hybridization-based binding of the nucleic acid molecules to the oligonucleotides on the target surface;
or if the nucleic acid molecules are attached to the sample
a) providing the sample containing nucleic acid molecules located either on the surface or within the sample;
b) providing a target surface with immobilized oligonucleotides;
c′) providing conditions for releasing the nucleic acid molecules;
d) assembling the sample and the target surface with a medium in between sample and target surface;
e) providing conditions for diffusion of the nucleic acid molecules from the sample to the target surface and hybridization-based binding of the nucleic acid molecules to the oligonucleotides on the target surface;
or
a) providing the sample containing nucleic acid molecules located either on the surface or within the sample;
b) providing a target surface with immobilized oligonucleotides;
d) assembling the sample and the target surface with a medium in between sample and target surface;
c′) providing conditions for releasing the nucleic acid molecules;
e) providing conditions for diffusion of the nucleic acid molecules from the sample to the target surface and hybridization-based binding of the nucleic acid molecules to the oligonucleotides on the target surface.

Optionally the method according to the invention comprises after step e) further step f):

f) providing conditions for slowing down the formation of new hybrids of nucleic acid molecules and oligonucleotides.

Another optional step comprises: disassembling the sample and target surface. If this step is part of the inventive method it will be the last step of the inventive method, which means it may follow step e) or step f).

Consequently, the present invention refers to a method for production of replica of of nucleic acid molecules not attached to a sample, comprising the following steps:

a) providing the sample containing nucleic acid molecules located either on the surface of the sample or within the sample;
b) providing a target surface with immobilized oligonucleotides, capable to hybridization-based binding to the nucleic acid molecules; and
bc) providing conditions to minimize shift of the nucleic acid molecules from the original positions; and
d) assembling the sample and the target surface with a medium in between; and
e) providing conditions for diffusion of the nucleic acid molecules from the sample to the target surface and hybridization-based binding of the nucleic acid molecules to the immobilized oligonucleotides.

The present invention refers further to a method for production of replica of nucleic acid molecules being attached to a sample, comprising the following steps:

a) providing the sample containing nucleic acid molecules located either on the surface of the sample or within the sample;
b) providing a target surface with immobilized oligonucleotides; and
c′) providing conditions for releasing of the nucleic acid molecules; and
d) assembling the sample and the target surface with a medium in between; and
e) providing conditions for diffusion of the nucleic acid molecules from the sample to the target surface and hybridization-based binding of the nucleic acid molecules to the immobilized oligonucleotides.

The present invention refers also to a method for production of replica of nucleic acid molecules being attached to a sample, comprising the following steps:

a) providing the sample containing nucleic acid molecules located either on the surface of the sample or within the sample;
b) providing a target surface with immobilized oligonucleotides; and
d) assembling the sample and the target surface with a medium in between; and
c′) providing conditions for releasing nucleic acid molecules from the original positions in the sample,
e) providing conditions for diffusion of the nucleic acid molecules from the sample to the target surface and hybridization-based binding of the nucleic acid molecules to the immobilized oligonucleotides.

It is preferred within the methods according to the invention that step a) reads as follows:

a) providing the sample containing inhomogeneous distributed nucleic acid molecules located either on the surface of the sample or within the sample.

That the distribution of the nucleic acid molecules within the sample or on the surface of the sample is inhomogeneous refers to samples wherein at least one type of nucleic acid molecule, which means one nucleic acid molecule having a specific sequence is not located in each area of the sample in the same concentration. Alternatively an inhomogeneous distribution occurs if at least one area of the sample differs in its nucleic acid molecules contained (at least one specific nucleic acid molecule is missing or at least one specific nucleic acid molecule is added compared to other areas of the sample).

Thus the inventive methods disclosed herein are especially useful if samples are provided on which or wherein an arbitrary number of nucleic acid molecules is contained but not in an evenly distributed manner or homogeneously distributed manner or a uniformly distributed manner, because one advantage of the present invention is that the information can be kept and can be obtained where each specific nucleic acid molecule was located in the sample as originally provided. Thus samples unlike fermentation media, waste water or urine are preferably used, wherein the presence or at least the concentration of the nucleic acid molecules which shall be detected is different depending on the location or area of the sample. Thus step a) in all methods disclosed herein could alternatively also read as follows:

a) providing the sample containing nucleic acid molecules located either on the surface of the sample or within the sample, wherein the presence or the concentration of the nucleic acid molecules varies depending of the area of the sample.

Step a) in all methods disclosed herein could alternatively also read as follows:

a) providing the sample containing nucleic acid molecules located either on the surface of the sample or within the sample, wherein the nucleic acid molecules are unevenly distributed over the surface of the sample or within the sample.

Step e) of the inventive method refers to incubating the assembly of the sample and the target surface of step d) under conditions sufficient to allow diffusion or migration of the nucleic acid molecules from the sample to the target surface and subsequently allow hybridization of the nucleic acids to the immobilized oligonucleotides. These conditions are explained in more detail above. During the inventive method lateral movements of the nucleic acids are suppressed so that the term “diffusion” or “migration” of the nucleic acid molecules in step e) refers only to a movement of the nucleic acid molecules primarily along a perpendicular axes. Thus the nucleic acid molecules leave the sample on a vertically way, on the direct route, to the target surface so that on the surface of the target a copy or replica is created which contains the nucleic acid molecules in an unaltered relative distribution or at least in a relative distribution with a minimal distortion.

The term “sample” as used herein refers to an object with a two or three-dimensional distribution of nucleic acid molecules. Thereby the consistence of the sample has to be in such a way that the nucleic acid molecules of interest have preferably an inhomogeneous distribution which is not highly variable. Thus, the nucleic acids should not be in solution and should not be able to freely diffuse within the sample. Preferred samples are non-fluidic, gel-like, fixated or solid. Examples of suitable samples are tissue sections, tissue blocks, a gel layer, a cell, a cell layer, a tissue array, yeasts or bacteria on a culture plate, membrane, paper or fabric, or a carrier with spots of isolated or synthetic nucleic acid molecules. In general the sample may comprise a carrier made of glass, plastic, paper, a membrane (e.g. nitrocellulose) or fabric. For example a tissue section is usually applied on a glass slide. A cell layer could also be provided on a glass slide or on a plastic dish. Unicellular organisms may be provided on culture plates, on filter paper or on a fabric. The nucleic acid molecule may be within the sample for example within a fixed cell, within a gel or within a tissue. Alternatively the nucleic acid molecules may be provided on the surface of a sample like a microarray (2D array on a solid substrate; usually a glass slide or silicon thin-film cell), preferably a DNA array also commonly known as DNA chip or biochip. Most preferable the sample is a tissue section. Said tissue section but also other samples (e.g. cells or unicellular organisms) may be frozen, (fresh frozen or fixed frozen) fixed (formaldehyde fixed, formalin fixed, acetone fixed or glutaraldehyde fixed) and/or embedded (using paraffin, Epon or other plastic resin). Such tissue sections like can be prepared with a standard steel microtome blade or glass and diamond knives as routinely used for electron microscopic sections. Furthermore small blocks of tissue (less than 15 mm thick) can be processed as whole mounts. In case the nucleic acid molecules are on the surface of the sample, thickness of the sample does not really matter so that any thickness could be used. In case the nucleic acid molecules are located within the sample like tissue slides, thickness should be in a range that the nucleic acid molecules could move out of the sample to the target surface. A preferred thickness of such samples is for example 1 μm to 1 mm.

The nucleic acid molecules can be either located on the surface of the sample or within a layer of the sample. Preferably, the nucleic acid molecules located on the surface are distributed on a nucleic acid array or protein array, and the nucleic acid molecules distributed within the sample are preferably distributed in a gel layer, in tissue section, in cell or tissue array or in block of tissue. For example, the nucleic acids can be contained in a gel and can be mobilized out of the gel to the surface of the gel. Alternatively, the nucleic acids can be provided on a glass slide.

The sample with nucleic acid molecules also comprises nucleic acids that are hybridized to the nucleic acids on the sample. This means that nucleic acids could be distributed on the sample and to this nucleic acids further nucleic acids are hybridized. Thus providing a sample with nucleic acids molecules located either on a surface or within a layer also includes hybridization products of nucleic acids. Consequently, the term nucleic acids also comprise hybridization products of nucleic acids.

The term “medium” as used herein refers to any material which allows nucleic acid molecules to diffuse through. Hence the term “medium” includes solutions, gels as well as other viscous or honey-like materials. Most preferably the medium used within the inventive method is a solution which may be an aqueous solution like a buffer, preferably on basis of PBS-buffers (Phosphate buffered saline) as well as Tris- and triethanolamine buffers (TE-buffer). It is further preferred that the pH-value of the used medium prevents denaturation of the nucleic acid molecules. Hence the pH of the medium or buffer is most preferably adjusted around 7.5 for RNA and around 8.0 for DNA. The medium or solution may further comprise some additives like cleavage agents (enzymes) or inhibitors of RNase or Dnase. Thereby the medium in the assembly of the sample and the target surface can also be emitted by the sample or the target surface. For example if the sample is a gel or contains a gel on the surface the medium may be a thin liquid film which is generated when some liquid leaks out of the gel due to some pressure during the assembling of the sample and the target surface.

The medium used in the inventive method should be chosen such that the nucleic acid molecules from the sample can reach the target surface by diffusion through the medium. The medium is used for diffusion of nucleic acid molecules from the sample to the target surface. This medium is preferably a liquid layer. Viscosity of the liquid layer may be increased to minimize the liquid flow along the target surface, for example, by inclusion of polymer molecules into the liquid. In the extreme case, those polymers may form a gel, which completely prevents the liquid flow, but preserves a possibility to nucleic acid molecules to diffuse from the sample to the target surface.

Step d), assembling the sample and the target surface with a medium in between comprises that the target surface is placed on top of (or below, depending on the direction of the transfer) the sample wherein the medium is added to the sample or to the target surface before. Assembling of the sample and the target surface in step d) is preferably done in such a way, that the distance from positions of the nucleic acid molecules on the surface of the sample or within the sample to the target surface is smaller than the distortion acceptable for the replica. This means if the tolerable or acceptable distortion is less than 1 mm the distance between the sample and the target surface should most preferably be less than 1 mm.

However this is a question of resolution and in case a high resolution is desired, the distance between sample and target surface should be less or much less than the distortion. Since the degree of distortion is a question of resolution provided by the inventive methods, step d) in all methods disclosed herein could also read as follows:

d) assembling the sample and the target surface with a medium in between sample and target surface in a way that the distance between sample and target surface is minimized.

or step d) in all methods disclosed herein could alternatively read as follows:

d) assembling the sample and the target surface with a medium in between sample and target surface in a way that the distance of each nucleic acid molecule is less than the distortion of the respective nucleic acid molecule.

or step d) in all methods disclosed herein could alternatively read as follows:

d) assembling the sample and the target surface with a medium in between sample and target surface in a way that the distance each nucleic acid molecule has to move in straight direction to the target surface is less than the distance the respective nucleic acid molecule is allowed to move straight in a direction perpendicular to the direction which is straight to the target surface.

However since the distortion is only an aspect how accurate the obtained data are but not whether the methods disclosed herein work, step d) could in all methods disclosed herein also simplified as followes:

d) assembling the sample and the target surface with a medium in between sample and target surface
or like
d) assembling the sample and the target surface with a medium in between sample and target surface so that the nucleic acid molecule can move to the target surface.

The term “distortion” can also be explained as the drift of the nucleic acid molecules.

If the sample consists or comprises of a layer the maximal possible distance of the nucleic acid molecules in the sample to the target surface should be smaller than the distortion acceptable for the replica. Therefore the distance from the surface of the layer not facing the target surface (or the bottom side) is relevant. “Distortion” as used herein denotes the alteration of the original, relative distribution of the nucleic acid molecules during the inventive method. One aim of the inventive method is to avoid distortion or at least to lessen distortion up to a tolerable extent.

Furthermore the medium used prevent the direct contact of the sample and the target surface, which is important for prevention of contamination of the target surface because of unspecific binding. Of course a direct contact of the sample and the target surface should also be avoided during assembling and disassembling of the sample and the target surface.

When the nucleic acid molecules are distributed on a two-dimensional surface, such as a glass slide (nucleic acid or protein array) there is no doubt, that they are a two-dimensional object, which can be replicated on another two-dimensional surface. But there are a lot of significant biological entities, which have three-dimensional nature, but should be used for creating of useful two-dimensional replicas. In some cases (molecules in a gel after electrophoresis) only two-dimensional distribution have sense, and the third dimension may be ignored completely. In other cases (molecules in a tissue section or on the surface of block of tissue) it is possible either to ignore the third dimension (in a case of tissue sections) and analyze two-dimensional distribution of molecules, or to make a replica of molecules located in a thin but three-dimensional surface layer (block of tissue). For preparation of replicas of molecules located in a layer, it is possible either (i) to use passive diffusion of nucleic acids molecules to the surface of the layer, or (ii) to organize directional migration using liquid flow (blotting) or electric field (electrophoresis), or (iii) to destroy or solubilize the layer and convert it into two-dimensional object (solubilization of the gel, proteinase digestion or lysis of tissue section).

The target surface comprises a plurality of at least one type of oligonucleotides attached to the target surface. The target surface can be of any texture. Within the inventive methods various materials may be used for preparation of targets and target surfaces. Preferably the target surface is a surface of glass, plastic, metal, paper, or porous membrane, which may be covered with a gel, dendrimers or microbeads and wherein the oligonucleotides on said target surface are made of DNA, RNA, LNA, PNA or mixture or hybrids of those and immobilized on the target surface by covalent or non-covalent binding directly to the surface or through gel, dendrimers or other chemical compounds attached to the surface. Porous materials like porous membranes (i) permit organization of directional migration of molecules through them either using liquid flow (blotting) or electric field (electrophoresis), besides (ii) they give a possibility to change the composition of the medium between sample and target surface during replication without disturbing sample-target surface assembly. Porous membranes and supports covered with gel, dendrimers or microbeads usually have a higher binding capacity for immobilization of oligonucleotides.

The target surface according to the invention should be covered with oligonucleotides, at least in the area to which the transfer is performed. Oligonucleotides on a target surface used in the inventive methods are DNA, RNA, LNA, PNA or mixture of those. A lot of methods are known in the prior art for immobilization of oligonucleotides which are usable within the present invention: covalent or non-covalent binding directly to the surface or through gel, dendrimers or other chemical compounds attached to the surface. More than one type of oligonucleotides may be immobilized on the target surface. Different oligonucleotides may be immobilized as a mixture (for example, for replication of several loci in parallel) or in different areas of the target surface (for example, for replication of microarrays, Example 7, FIG. 12). For immobilization of area-specific oligonucleotide on the target surface it is possible to use such methods as spotting, on-surface synthesis, bead immobilization, etc.

Transferred nucleic acid molecules hybridize preferably directly to the oligonucleotides immobilized on the target surface (FIG. 1A). But in one embodiment of the present invention hybridization-based binding occurs through adapter oligonucleotides which are complementary both to the nucleic acid molecules from the sample and to the oligonucleotides on the target surface (FIG. 1B). These adapter oligonucleotides are characterized by at least two regions, wherein one region is at least partially complementary to a nucleic acid on the sample and another region is at least partially complementary to the oligonucleotides attached to the target surface. In this embodiment the nucleic acids do not hybridize directly to the at least one type of oligonucleotides on the target surface but said hybridization-based binding occurs through adapter oligonucleotides. Adapter oligonucleotides allow using the same target surfaces for hybridization probes with different regions responsible for binding to the target surface. The term “adaptor oligonucleotide” or “fusion oligonucleotide” as used herein refers to an oligonucleotide that consist of a first sequence portion able to hybridize to at least one type of oligonucleotide on the target surface followed by a sequence able to bind nucleic acid molecules on or in the sample. Thereby the adaptor oligonucleotides are able to mediate binding of the nucleic acid molecules to the target surface.

Hence it is preferred within the inventive method that in step e) the hybridization-based binding occurs through adapter oligonucleotides which are complementary both to the nucleic acid molecules from the sample and to the immobilized oligonucleotides on the target surface.

The method of invention does not require a direct contact between the nucleic acids distributed in the sample and the oligonucleotides on the target surface. Nucleic acid molecules diffuse through the liquid layer from the sample to the target surface. Also, some diffusion along the surface occurs during diffusion of molecules from the sample to the target surface. Diffusion along the surface leads to some distortion of relative positions of molecules after replication—blurring. To minimise distortion the liquid layer should be as thin as possible, so the surfaces in the assembly and preferably in the sandwich-like configuration should be tightly pressed to each other. Assembling such as sandwich-like configurations can be performed as shown in FIG. 2. It is impossible to set up the maximum acceptable distance between sample and target surface for all possible replications, because this distance depends on the acceptable level of distortion. It is desirable, that the distance between sample and target surface would be smaller than acceptable size of distortion.

The terms “sandwich-like configuration” or “assembly” both refer to the configuration that the sample and the target surface are brought into contact with each other with thin liquid layer or solution in between.

It is desirable, that nucleic acid molecules do not go off their positions in the sample during preparation of sandwich-like assembly. There are three ways to organize molecular transfer between the sample and the target surface within the inventive methods:
nucleic acid molecules are free or released before preparation of sandwich-like assembly;
nucleic acid molecules are fixed to the sample. Release is started just before preparation of sandwich-like assembly and proceeds after sandwich-like assembly is ready;
nucleic acid molecules are fixed to the sample and released only after sandwich-like assembly is ready.

The nucleic acid molecules may be either attached or not attached (free) to the sample. Attached molecules does not change their positions. It is convenient before and during assembling of sample and target surface, because molecules keep the same distribution. But it is impossible to organize transfer of attached molecules from the sample. Attached molecules should be released from their positions to make diffusion from the sample to the target surface possible.

Even not attached nucleic acids may keep their original positions for some time. Free diffusion of nucleic acid molecules within the sample may be physically hindered by surrounding matrix, for example by agarose or acrylamide polymers in gels or by cell components in tissue sections

In preferred embodiments of the present invention nucleic acid molecules are free (not attached) or released before preparation of sandwich-like assembly. In the case of not attached or not fixed molecules it is preferred to apply conditions to minimize their shift from original positions during preparation of sandwich-like assembly. These conditions may be: release molecules immediately before assembling, fast assembling, insertion into the sample of a net which restricts migration of molecules, decrease of temperature, assembling on a temperature low enough to slow down a shift of nucleic acid molecules from the original positions. Preferably, the temperature is decreased below 20° C., more preferably 16° C., preferably 12° C., even more preferred 8° C., and more preferred 4° C. When sandwich-like assembly is ready the temperature of the sample can be increased again to speed up the diffusion of nucleic acid molecules from the sample to the target surface. Therefore it is preferred that assembling the sample and target surface in step d) of the inventive method is performed with a temperature low enough to slow down a shift of the nucleic acid molecules from the original positions on or within the sample.

On the other hand if the nucleic acids are attached to the sample it may be advisable to apply conditions, wherein gradual release of the nucleic acids in the sample occurs. The nucleic acids may be attached to the sample, for example by hybridization to complementary sequences covalently bound to the sample, or through cleavable groups.

In another embodiment release is started just before preparation of sandwich-like assembly and proceeds after sandwich-like assembly is ready. Thereby nucleic acid molecules in the sample are held on the original positions by chemical- or enzyme-sensitive binding and said conditions for releasing of nucleic acid molecules from the sample in step c′) are provided by a cleavage agent which destroys the said binding and acts slow enough to ignore those molecules which change the position before assembling sample against the target surface in step d). Releasing of nucleic acid molecules from the sample by the cleavage agent may be slowed down by decreasing concentration of said agent or by providing reaction conditions suppressing the activity of said agent at least partially. The main idea is to release most of the nucleic acid molecules when the assembly is ready. If the activity of the cleavage agent is the same during assembling and after assembly is ready the time of transfer should be significantly longer, than the assembling time. Another option is to increase activity of the cleavage agent when assembly is ready. It is possible to use such approaches as:
slow incorporation of cleavage agent (or its cofactor) into the sample;
regulation activity by temperature or by change of solution.

Preferred are an inventive method, wherein nucleic acid molecules in the sample are held on the original positions by chemical- or enzyme-sensitive binding and said conditions for releasing of the nucleic acid molecules in step c′) are provided by a cleavage agent which destroys said binding and acts slow enough to ignore those molecules which change the positions before assembling sample against the target surface in step d). It is further preferred that the releasing of nucleic acid molecules by the cleavage agent is slowed down by decreasing the concentration of the cleavage agent or by providing reaction conditions suppressing the activity of the agent at least partially.

Thus before assembly of the sample with the target surface conditions can be applied, wherein gradual release of the nucleic acid molecules from the sample occurs. Said conditions for gradual release of nucleic acid molecules from the sample may be the cleavage agent which acts slow enough to ignore those molecules which change the position before assembling sample against the target surface. In one embodiment said low activity of the cleavage agent is provided by decreasing concentration of the said agent or by providing reaction conditions decreasing the activity of the said agent.

Diffusion of nucleic acid molecules within the sample may be physically hindered by surrounding matrix, for example agarose or acrylamide gel. In this case diffusion exists but it is very slow: the time of appearing of free molecules on the surface of the sample is much longer than the time of assembling sample/target surface sandwich. It is possible to assemble a sandwich and wait till nucleic acid molecules diffuse enough to reach the target surface. It might be possible to speed up the diffusion by raising the temperature of the sandwich. Nucleic acid molecules may be just physically stuck within the sample, for example in gel after gel electrophoresis.

The sample with nucleic acid molecules and the target surface are assembled under conditions where nucleic acid molecules do not go off their positions on the sample surface. Such conditions can comprise e.g. low temperature, a filter or net between the surfaces, enzymes and/or chemical substances preventing detachment at this stage or vice versa the lack of such enzymes and/or chemical substances needed for detachment.

Preferably the sample with nucleic acid molecules and the target surface are assembled under “wet” conditions meaning that the sample and target surface are surrounded by solution, i.e. liquid and/or that liquid is between both surfaces. Both surfaces are arranged such that both surfaces come into contact with each other in a sandwich-like configuration. A thin liquid film can exist between both surfaces. The liquid between the surfaces and/or around the assembled sandwich-like configuration can comprise enzymes and/or chemical substances needed e.g. for detachment. If a filter or net between the surfaces is used during assembly, such a net would prevent direct contact of the surfaces.

The surfaces in the sandwich-like configuration shall be tightly pressed to each other to make the distance between the surfaces so that the distance between both surfaces is so small that no blurring of the distribution pattern occurs. Assembling such sandwich-like configurations is performed as shown in FIG. 2 and is well known to the skilled artisan and corresponds mutatis mutandis to the procedures known from e.g. western/northern blotting. Surface assembly would be done preferably at room or lower temperature, so that the nucleic acid molecules do not go off the sample surface. Generally, the inventive method does not require a direct contact between the nucleic acids distributed on the sample and the oligonucleotides on the target surface. This means that transfer may be performed between large solid surfaces, which can't form uniform tight contact.

The terms “sandwich-like configuration” or “assembly” both refer to the configuration that the sample and the target surface are brought into contact with each other.

The incubation time is dependent from many variables, such as accessibility of the nucleic acids on the sample, incubation temperature and other factors. Generally, incubation time should be long enough to allow sufficient hybridization, but still short enough to prevent e.g. unspecific binding. Under aspects of process economy, the incubation time should be chosen to be as short as possible. The skilled artisan can determine the optimal incubation time with minimum routine experimentation.

In another embodiment nucleic acid molecules are fixed to the sample and released only after sandwich-like assembly is ready.

Detachment conditions (certain temperature, light, solution) may be applied to the assembly of the sample with distributed nucleic acid molecules and the target surface. Temperature may be applied to release nucleic acid molecules if the binding to the sample is temperature-sensitive. Thus, in one embodiment the condition for releasing the nucleic acid molecules from the original positions in the sample occurs by increasing the temperature.

One of the convenient instruments for providing conditions for releasing the nucleic acid molecules from the original positions in the sample without disturbing the assembly is increasing of the temperature. Therefore it is preferred that the condition for releasing the nucleic acid molecules from the original positions in the sample in step c′ comprises increasing the temperature In another embodiment the nucleic acid molecules are held on the original positions in the sample by temperature-sensitive binding or the medium comprises a thermoactivated cleavage agent. It is further preferred that the sensitive bindings are done by hybridization, or through thermolabile covalent bonds, abasic sites or formaldehyde linkages or wherein the thermoactivated cleavage agent is an enzyme. Activity of a lot of enzymes strongly depends on the temperature, so those enzymes may be used as thermoactivated cleavage agents. Detachment can also occur by providing a thermoactivated cleavage agent, enzyme or chemical reagent in the solution between the sample and the target surface. Thus a change in the temperature after assembling may release nucleic acid molecules which have been fixed or attached to the sample before.

Another instrument for providing conditions for diffusion of nucleic acid molecules from the sample to the target surface without disturbing the assembly is changing the solution between the sample and the target surface. To preserve the assembly intact during changing of the solution the sample or the target surface or both should be permeable for liquid. Therefore another preferred embodiment refers to a method wherein the condition for releasing the nucleic acid molecules from the original positions in the sample in step c′) comprises changing the medium between the sample and the target surface.

Hence, in another embodiment the condition for releasing the nucleic acid molecules from the original positions in the sample is changing the solution between the sample and the target surface.

The possibility to change solution in the contact area in the assembly substantially increases the number of variants of nucleic acid molecules attachment to the sample, and consequently, types of samples. If nucleic acid molecules are attached to the nucleic acid sample by hybridization to a complementary sequence, duplex may be denatured by changing the pH or ionic strength of the solution, or changing the solution to the one decreasing the denaturation temperature (like formamide). Nucleic acid molecules may be attached through some cleavable group. The cleavage agent (e.g. enzyme or chemical substance) may be delivered after the sandwich assembly. If nucleic acid molecules are held on the original positions in the sample by hybridization then said new solution should destabilizes hybridization by changing pH or ionic strength of the solution or by decreasing the melting temperature of the duplex like formamide. If nucleic acid molecules are held on the original positions in the sample by chemical- or enzyme-sensitive binding then said new solution should contain a cleavage agent.

In one embodiment the nucleic acid molecules are held on the original positions in the sample by hybridization and a new medium destabilizes hybridization by changing pH or ionic strength of the medium or by decreasing the melting temperature of the duplex like formamide, or the nucleic acid molecules are held on the original positions in the sample by chemical- or enzyme-sensitive binding and the new solution contains a cleavage agent, and wherein either the sample or the target surface or both are permeable for the medium and during changing of the medium the assembly remains intact.

If nucleic acid molecules are attached to the sample by hybridization to a complementary sequence, duplexes may be denatured by heating the assembly. Nucleic acid molecules may be covalently attached to the sample through thermolabile bonds like abasic site or formaldehyde linkages. In such cases heating would destroy the binding. Binding may also be organized through enzymatically or chemically cleavable site, where cleavage enzyme or chemical reagent should be thermoactivated. Cleavage agent should then be present in the solution, but during assembling the sandwich it should not act (e.g. to prevent working of an enzyme sandwich may be assembled at low temperature) or should act slowly (e.g. low concentration, inappropriate temperature).

Light-sensitive reactions are another instrument for providing conditions for diffusion of nucleic acid molecules from the sample to the target surface without disturbing the assembly. Nucleic acid molecules should be held on the original positions in the sample by photocleavable binding and either the sample or the target surface should be transparent for the light with required wavelength. Hence, in one embodiment the condition for releasing the nucleic acid molecules from the original positions in the sample in step c′) comprises light and wherein the nucleic acid molecules are held on the original positions in the sample by photocleavable binding and wherein either the sample or the target surface are transparent for the light with required wavelength. Sandwich should be assembled without the activating light.

Some diffusion along the target surface occurs during diffusion of the nucleic acid molecules from the sample to the target surface. Diffusion along the target surface leads to distortion of relative positions of molecules after replication, also called blurring. Within the inventive method distortion should be as small as possible or at least as small as the user can accept. We described previously one measure to prevent such distortion, namely minimizing the distance between the sample and the target surface. The second measure for prevention of distortion is to subdivide the sample, the target surface or both into isolated regions, wherein the nucleic acid molecules can't cross the borders of said regions. If both the sample and the target surface are subdivided it is self-evident that they should be divided into corresponding regions or areas which means that the areas or regions are congruent. Isolated regions restrict blurring or distorsion, because diffusion of the nucleic acid along the target surface is restricted by the borders of the isolated regions. Isolated regions or areas may be created by using a mask with isolated holes or by scratching the sample or the target surface. Mask with holes may be located between the sample and the target surface. It is even better, if the mask is pressed into the sample to split the sample in a number of isolated regions or areas. Besides, the mask may prevent the direct contact of the sample and the target surface, which is important for prevention of contamination of the target surface because of unspecific binding. Scratching may be used to create borders of isolated regions by exposing of hydrophobic basis of the sample or of the target surface. Therefore it is preferred that the sample, the target surface or both are subdivided into isolated regions, wherein the nucleic acid molecules can't cross the borders of said regions and wherein the regions are created by using a mask with isolated holes or by scratching the sample or the target surface.

A third possibility to prevent distortion is to facilitate diffusion into the direction of the target surface by liquid flow (blotting) or by electric field (electrophoresis). For the directional transfer both the sample and the target surface should be permeable for the liquid flow or conductive for electric current. Therefore one preferred embodiment comprises that the conditions for diffusion of nucleic acid molecules from the sample to the target surface are facilitated by liquid flow (blotting) or by electric field (electrophoresis).

One further advantage of hybridization-based replication is the possibility to significantly slow down the formation of new hybrids of nucleic acid molecules on the target surface before disassembling of the sample and the target surface by decreasing the temperature close to 0° C. It prevents attaching of nucleic acid molecules to the wrong places on the target surface when the sandwich-like structure is disturbed. Besides, low temperature generally slows down activity of cleavage agents and the speed of the diffusion of the nucleic acid molecules from the sample. Thus, in one embodiment of the invention the conditions for slowing down the formation of new hybrids of the nucleic acid molecules before disassembling the sample and the target surface are carried out by decreasing of the temperature of the sample or the target surface, the medium in between or all of them.

Under certain conditions it may be necessary to wash the target surface after replication. Washing can be performed with known washing buffers, such as PBS, TE or any other washing buffer known to the skilled artisan. Care should be taken not to use washing buffer, which are able to disrupt the bonding between the hybridized nucleic acid molecules and their complementary sequences. Optionally, washing of the target surface may be performed at low temperature.

The target surface may be used for amplification of transferred molecules: bridge amplification of molecules with special flanking adaptor regions or RCA amplification of circular molecules (FIG. 4). Corresponding clones are located in the same positions as transferred molecules. Amplified copies of original population of nucleic acid molecules may be used for ex situ hybridization and for preparation of copies of microarrays.

An “oligonucleotide” as used herein is a short nucleic acid polymer, typically with fifty or fewer bases. Although for the purposes the present invention, the oligonucleotides can have more or less nucleic acids.

Before separating the surfaces, it may be necessary to decrease the temperature close to 0° C. At low temperature hybridization speed becomes low, which prevents attaching of nucleic acids to the wrong places on the target surface when the sandwich-like configuration is disturbed. Optionally, washing of the target surface may be performed at low temperature. Thus, in one embodiment before disassembling the sample and the target surface slowing down formation of new hybrids of nucleic acid molecules is done by decreasing the temperature of the assembly.

In one embodiment a plurality of adapter oligonucleotides is provided. The adapter oligonucleotides are complementary both to the nucleic acid molecules from the sample and to the nucleic acid molecules on the target surface. These adapter oligonucleotides are characterized by at least two regions, wherein one region is at least partially complementary to a nucleic acid on the sample and another region is at least partially complementary to the oligonucleotides attached to the target surface. In this embodiment the nucleic acids do not hybridize directly to the at least one type of oligonucleotides on the target surface but said hybridization-based binding occurs through adapter oligonucleotides which are complementary both to the nucleic acid molecules from the sample and to the nucleic acid molecules on the target surface.

The general mechanism is shown in FIG. 1B in comparison to direct hybridization of the nucleic acids to the target surface as shown in FIG. 1A. The use of adapter oligonucleotides allows to use the same target surfaces for hybridization probes with different regions responsible for binding to the target surface.

After the nucleic acids from the sample have been transferred to the target surface enzymatic reactions may be performed with the replica on the target surface, wherein said enzymatic reactions include primer extension, ligation, rolling circle amplification, in situ PCR amplification, bridge PCR amplification, sequencing, restriction (see FIGS. 3, 4 and 13).

In yet another embodiment the nucleic acid molecules in the sample or nucleic acid molecules on the target surface contain known sequences, which (optionally) get inserted in the nucleic acid molecules from the target surface or nucleic acid molecules from the sample by primer extension or ligation reactions and said known sequences are further used for analysis of replicas, wherein said analysis may be performed on the target surface or in solution.

In another embodiment the said known sequences serve to distinguish the samples, target surfaces, replication experiments, wherein said known sequences are different between the samples, target surfaces, replication experiments or to determine the position of nucleic acid molecules on the target surface or in the sample and wherein said known sequences are different in different regions of the sample or of the target.

Oligonucleotides on the target surface may contain besides the regions for hybridization-based binding of nucleic acid molecules from the sample, sequences for labeling the transferred nucleic acid molecules. Such sequences get attached to the transferred nucleic acid molecules or their derivatives (extention, ligation products) after replication by ligation or primer extension. In the following analysis of the replicated molecules or their derivatives, for example by sequencing or hybridization, the labeling sequence would reveal to which oligonucleotide a certain replicated molecule was bound.

Labeling sequences may be used for position coding of the transferred nucleic acid molecules. For example, target surface may be divided into a number of small regions (code regions), oligonucleotides in each region containing unique nucleic acid codes—a 4-100 nt nucleic acid sequence. Coding target surface may be used for position coding of transferred nucleic acid molecules: in each code region a different nucleic acid code will be added to the nucleic acid molecules. Adding may be performed by for example ligation, primer extension in appropriate conditions. By adding position-specific codes, information about surface coordinates of nucleic acid molecules is recorded in the sequences of nucleic acid codes. It is then possible to remove the coded replicated nucleic acid molecules from coding surface into solution. In the course of further analysis reading of the codes gives information about original positions of nucleic acid molecules.

In these embodiments the hybridization probes are transferred to a target surface with preformed coded regions—thus, hereinafter named coding surface—and coding oligonucleotides already distributed on the coding surface.

The general procedure is that prior to transfer of the nucleic acids from the sample to the coding surface, so called code regions are created on the coding surface. Thus the coding surface is subdivided in any number of code regions, the number of code regions being dependent on the desired resolution. The code regions can be created physically, by applying e.g. a filter or net on the original surface, wherein each “hole” in this net or filter would represent one code region. It is also possible to use beads with coding oligonucleotides attached to them, wherein each bead would correspond to one coding region. However, it is also possible that the code regions are not created physically but only imaginary code regions are created. This could be realized by e.g. registering the coordinates of each code region on the sample. The coding surface comprises a plurality of coding oligonucleotides attached to the target surface. As long as the coding surface can bind oligonucleotides to its surface, the coding surface can be of any texture. The coding surface consists of code regions in each code region coding oligonucleotides have a different nucleotide code. The more precise localization of transcripts is required, the smaller code regions should be used. The more code regions should be on the coding surface—the longer code regions are required to have a unique code in each code region.

Such coding surface may be prepared for example by spotting nucleic acid codes, by making layer of beads with nucleic acid codes, by synthesizing nucleic acid codes directly on the surface.

The transferred nucleic acids would be coded using primer extension reaction: depending on the unique nucleic acid sequence in the coding oligonucleotides, nucleic acids will be extended with a certain unique sequence. The primer extension mix would contain nucleotides and polymerase in an appropriate buffer. Care has to be taken that during primer extension reaction, the nucleic acids do not go off their locations. Therefore, extension should be performed at temperatures below annealing temperature of the nucleic acids.

The result of the extension would be a double-stranded molecule, in which both stands have flanking regions required for sequencing and unique nucleic acid sequence from the coding oligonucleotides, required for revealing the original position on the original surface. The coded extension products can be removed from the double-stranded molecule by different methods. In one embodiment the coding surface is rinsed high-temperature (˜95° C.) solution. At high temperature, the double strands will be denatured and the non-covalently attached stands go into solution. Also high temperature inactivates the enzyme used for primer extension, so that no primer extension is possible in the solution.

In another embodiment, the coding oligonucleotides on the coding surface would further comprise a cleavable group. Due to this cleavable group, the whole double strand can be removed from the coding surface after destroying the cleavable group. The double strand may be further amplified and then sequenced.

It should be taken into consideration, that during transfer of nucleic acid molecules to the coding surface and during adding of nucleic acid codes to the nucleic acid molecules, nucleic acid codes should stay within the coding regions. Depending on the way of attachment of coded replicated nucleic acid molecules to the coding surface, nucleic acid molecules may be released independently from non-used nucleic acid codes or together with them. For example, when coded nucleic acid molecules are attached to the coding surface by hybridization, and nucleic acid codes are covalently attached, nucleic acid molecules may be released from the surface by denaturizing conditions, and nucleic acid codes will remain on the surface. When both nucleic acid codes and coded replicated nucleic acid molecules are attached to the coding surface in the same way, they will be released together. In the latter case nucleic acid codes either remain in the mixture with coded nucleic acid molecules if they do not interfere with further operations, or they would be removed, for example by size selection.

The present invention is also directed to a coding surface with a plurality of coding regions, wherein the coding surface is covered with a plurality of coding oligonucleotides, wherein the coding oligonucleotides are characterized by a 3′ part common to all coding oligonucleotides, and an individual nucleotide sequence of 4-100 nucleotides, characterized in that each coding region is covered only with coding oligonucleotides with the same individual nucleotide sequence of 4-100 nucleotides.

Hybridization-based replicas prepared according to the inventive method may be used for analysis of the transferred molecules, in particular for the sequencing of the nucleic acid molecules transferred from the sample. After replication sequencing is preferably performed directly on the target surface and the relative positions of the sequenced nucleic acid molecules resemble spatial distribution of the nucleic acid molecules in the original sample.

Hybridization-based replicas are especially useful for analysis of tissue sections. Tissue sections are important objects with two-dimensionally distributed nucleic acid molecules. Sequential sections restore a 3D spatial location of molecules. There are a lot of molecular biology methods, for example sequencing, which cannot be performed directly in tissue sections. It would be advantageous to transfer molecules from the tissue section to another surface, where appropriate methods of analysis could be performed.

Sequencing of nucleic acids replicated from the tissue sections is useful for example for expression profiling (Example 8) and for locus specific sequencing (Example 9). Expression profiling permits to analyze distribution and expression level of a number of genes in parallel on a single tissue section. Locus specific sequencing permits to analyze mutation status of a number of genes (for example, the state of oncogenes in a tumor) for all cells in a tissue section.

In the following some preferred methods are described in more detail.

Currently, transcripts in tissue sections are analyzed by in situ hybridization. Main restriction of this approach is the limited number of transcripts which it is possible to analyze simultaneously. The reason is that it is impossible to select considerable number of distinguishable labels for hybridization probes. Transcripts in tissue sections may be analyzed by sequencing and ex situ hybridization as follows:

In the second generation sequencing (SGS) platforms sequencing is performed on the surface of a special flowcell for millions of templates in parallel. 2D flowcell surface is similar to the slide with tissue section. Sequencing cannot be performed directly in the tissue section. However using a method of the invention it is possible to transfer the transcripts (hybridization probes, primer extension products) from tissue section to the surface of the sequencing flowcell preserving the distribution pattern.

The method may be conducted according the following flow chart:

Hybridization probes should have the structure as shown in FIG. 13A. Middle parts of probes are for hybridization to transcripts in tissue section. Flanking regions a and b are common for all probes and are required both for hybridization and sequencing on the SGS flowcell surface.

Hybridization probes may be selected to target from single to thousands of transcripts. They may be synthesized artificially or prepared from a sequencing library. To prevent unspecific hybridization of common parts of the hybridization probes in tissue section it is possible to reversibly block them with complementary oligonucleotides. These oligonucleotides should be removed before transfer of hybridization probes to the SGS flowcell surface.

Tissue section slide and SGS surface would be brought into tight contact, possibly with a net in between (see FIG. 2). The distance between surfaces (or the mesh size if a net is used) should be smaller than acceptable blurring of the distribution pattern. A net would also prevent direct contact of the tissue section and SGS surface.

Surface assembly would be done at room or lower temperature, so that the hybridization probes do not go off the surface. Detaching probes from the tissue section and attaching to the SGS surface would be regulated by the temperature. First, the temperature of the sandwich would be raised up to denaturize the hybridization probes. Then the temperature would be decreased to allow the common regions of hybridization probes annealing to the oligos immobilized on the SGS surface. In these conditions hybridization probes may hybridize back to the transcripts in tissue sections. However transcripts in the tissue section are few in comparison to oligos on the target surface, so probability to hybridize to the target surface is much higher than back to the tissue section.

The time of denaturation would be selected to allow enough probes to denature and move into solution between the surfaces. The time of hybridization should be adjusted so that enough but not too many probes are transferred to provide a necessary density of sequencing templates and so that probes do not diffuse too far away. Before separating the surfaces, the temperature would preferably be decreased close to 0° C. At low temperature hybridization speed becomes low, which prevents attaching of probes to the wrong places on SGS surface when sandwich is disturbed. Washing of the unhybridized probes from the SGS flowcell surface would be also performed at low temperature.

Amplification of the transferred probes on the SGS flowcell surface and further sequencing would be performed according to the known sequencing procedures. SGS would determine two parameters for each probe: (i) its partial or complete nucleotide sequence and (ii) position on the slide surface. Nucleotide sequence will identify which particular transcript was a target for a probe. Position of a probe on a flowcell will be set into correspondence with the position on the tissue section.

An alternative to SGS analysis is the analysis of transcripts in tissue sections by single molecule sequencing transfer of transcripts distribution pattern to the pattern of sequencing templates.

The procedure looks the same as described before, with the difference in sequencing approach: molecules transferred from the tissue section are sequenced directly by single-molecule sequencing approach, where transferred molecules are sequenced directly on the target surface with capturing oligonucleotides initializing the primer extension. Since no amplification on the target surface is required, only one type of oligonucleotides can be present on the sequencing surface for capturing sequencing templates by hybridization. This approach may be realized using single-molecule sequencing approach like for example that of Helicos. Single molecules sequencing allows for a higher density of sequencing templates.

A further alternative to SGS analysis is analysis of transcripts in tissue sections by ex situ hybridization. The procedure looks the same as described before but amplification of the transferred nucleic acid molecules on the target surface and removing of one strand. Then instead of sequencing, target surface is used for hybridisation with probes of interest. So, this is basically in situ hybridization but with targets transferred to another surface and amplified.

In situ amplification results in ˜1000 copies of transferred molecule. This allows increasing hybridization signal and thus sensitivity of transcripts analysis. Another advantage of this approach is that it makes possible to use same replica for several hybridizations with different probes without increase of background. Target molecules are covalently attached to the surface, so it is possible to use stringent conditions to wash off probes from previous hybridization. This increases the throughput of analysis in comparison to in situ hybridization.

Another preferred embodiment refers to marking positions of transcripts in tissue section by nucleic acid codes using a coding surface and subsequent analysis by SGS sequencing

This method allows to transfer transcripts (or corresponding to transcripts hybridization probes, primer extension products) from tissue section into solution and thereby preserving information about the distribution pattern. Molecules in the solution may be further processed according to standard sequencing protocols for sample preparation. Loading of sequencing flowcell would be performed as for standard sequencing library, so loading density will be even over the flowcell surface and adjustable. Having sequencing templates in the solution would also allow to use any SGS platform and thus be independent from the SGS surface.

The possible procedure of this preferred method is:

Middle parts of probes are for hybridization to transcripts in tissue section. Flanking regions are common for all probes and are required for hybridization to the coding surface (hybr. region) and sequencing on the SGS flowcell surface (seq. region 1). To prevent unspecific hybridization of common parts of the hybridization probes in tissue section it is possible to reversibly block them with complementary oligonucleotides. These oligonucleotides should be removed before transfer of hybridization probes to the coding surface.

The coding surface is covered with covalently attached coding oligonucleotides. The 3′ part, which is complementary to the hybridization region of the hybridization probes, is followed by code region. 5′ part is required for further sequencing on the SGS flowcell (seq. region 2). Coding oligo may be detached from the surface using a cleavage site. Cleavage site may be organised for example by a chemically, thermally or enzymatically destroyable nucleotide.

Coding surface consists of coding regions, in each region coding oligos have a different code part. The more precise localisation of transcripts is required, the smaller coding regions should be used. The more coding regions should be on the surface—the longer nucleotide sequence is required as a code.

Hybridized probes would be transferred to a coding surface as described before. Attachment to the coding surface would be realized by hybridisation of the hybridization region of the hybridization probes to the complementary 3′ part of the oligos on the coding surface. The result of the transfer would be a coding surface with hybridization probes attached to it in a mirror-distribution relative to the distribution of corresponding transcripts in tissues section.

Transferred hybridization probes would be coded using primer extension reaction: depending on the coding region, hybridization probe will be extended with a certain code sequence. Primer extension mix would contain nucleotides and polymerase in an appropriate buffer. Mix would be pipetted over the surface using for example HybriWell chambers from Grace Biolabs. It is important that during primer extension reaction, hybridization probes do not go off their locations. Extension should therefore be performed at temperatures below annealing temperature of hybridization region.

The result of the extension would be double-stranded molecules, in which both strands have flanking regions required for sequencing and code regions, required for revealing molecules position. Coded molecules can be removed from the slide and combined in the solution. This may be performed in two ways.

Variant 1. Coding surface would be rinsed in high-temperature (˜95° C.) solution. At high temperature, duplexes will be denatured and non-covalently attached strands will go into solution. Also high temperature would inactivate the enzyme used for primer extension, so that no primer extension would be possible in the solution (which may cause chimeric molecules formation). Single-stranded sequencing templates have common flanking regions required for SGS and may be further amplified or used directly for clonal amplification.

Variant 2. Duplexes would be removed from the coding surface after destroying of the cleavable group. Together with duplexes, non-extended coding oligos will also be removed from the coding surface, and may cause extension in solution, which may lead to wrong coding and formation of chimeric molecules. It is therefore necessary to pay attention that polymerase present in primer extention mix is washed away from the surface or inactivated prior to combining the duplexes in solution. Double-stranded sequencing templates may be further amplified or used directly for clonal amplification.

Further stages—amplification of the molecules, clonal amplification and sequencing would be performed according to the known SGS procedures (SOLiD platform from ABI; GA and HiSeq from Illumina).

SGS would determine two sequences for each sequencing template: (i) partial or complete transcript-specific sequence and (ii) sequence of the code. Code sequence will be set into correspondence with the distribution scheme of position coding primers on the tissue section slide, and reveal the initial position of the transcript in the tissue section.

Further preferred embodiment refers to marking positions of nucleic acids in tissue section with a sequenced SOLiD flowcell as a coding surface and subsequent analysis by Second Generation Sequencing (SGS), FIGS. 14, 15.

An already sequenced SOLiD flowcell is used as the coding surface. Clonally amplified sequencing templates are attached to the beads. After sequencing, position of each bead and sequence of molecules attached to it are known. Thus, sequences may serve as codes for hybridization probes transferred from tissue section slide.

Hybridization probes would have middle parts for hybridization to transcripts in tissue section. Flanking regions are common for all probes and are required for hybridization to the coding surface (hybridization region) and sequencing on the Illumina platform (illumination region 1). Hybridization region may hybridize to the common 3′ region (P2) of SOLiD sequencing templates. To prevent unspecific hybridization of common parts of the hybridization probes in tissue section it is possible to reversibly block them with complementary oligonucleotides. These oligonucleotides should be removed before transfer of hybridization probes to the coding surface.

Coding surface is a sequenced SOLiD flowcell: glass slide covered with beads. Each bead is a different code region. Unique middle parts of sequencing templates serve as codes. Hybridized probes would be transferred to the coding surface as described before. Attachment to the sequencing templates would be realized by hybridization of the hybridization region of the hybridization probes to the complementary P2 regions. The result of the transfer would be beads with hybridization probes attached to them.

Transferred hybridization probes would be coded using primer extension reaction: depending on the bead to which it is attached, hybridization probe will be extended with a certain code sequence. Primer extension mix would contain nucleotides and polymerase in an appropriate buffer. Mix would be pipetted over the surface using for example HybriWell chambers from Grace Biolabs. It is important that during primer extension reaction, hybridization probes do not go off their locations. Extension should therefore be performed at temperatures below annealing temperature of hybridization region. Sequencing templates would not be extended because in the course of the SOLiD sequencing protocol they are 3′ end blocked.

The result of the extension would be a hybridization probe to which the sequence of a SOLiD sequencing template is added, and which has a P1 sequence on 3′end. Coded molecules may be washed off the beads in denaturizing conditions and combined in solution. Single stranded coded molecules would be amplified to introduce illumination region 2 next to P1 part of the molecule. Result of amplification would be double-stranded molecules flanked with Illumina-platform specific illumination regions 1 and 2, which may be further amplified or used directly for clonal amplification and sequencing on the Illumina platform.

Illumina sequencing would determine two sequences for each sequencing template: (i) partial or complete transcript-specific sequence and (ii) sequence of the code. Code sequences will reveal the position of corresponding beads on the SOLiD flowcell and thus the position of original transcripts in the tissue section.

In the previous preferred methods described the aim was to reveal position of the nucleic acid molecules distributed within tissue section. For analysis of a panel of samples with 2D distributed nucleic acid molecules (e.g. cell arrays, tissue arrays) it may be necessary to reveal from which sample nucleic acid molecules originate. Previously described procedures work for these applications, too. If coding is used to mark nucleic acid molecules from a single sample, size of coding regions on the coding surface may be comparable to the size of a sample.

Replication method using holding nucleic acid molecules on the target surface by hybridization is highly selective. From a mix of nucleic acid molecules transferred from a sample with 2D distributed nucleic acid molecules to the target surface, only those will be replicated, which have a sequence complementary to the capturing oligonucleotides.

Selectivity may be used for example for selection of full-length oligonucleotides after on-surface synthesis. Oligonucleotides synthesis is performed from 3′ to 5′ end. For selection of full-length oligos it is possible to use 5′ sequences. In the FIG. 12A all synthesized oligonucleotides should have the same 5′ region. Oligonucleotides complementary to this region are attached to the target surface. During replication of synthesized oligos to the target surface, only full length oligonucleotides get captured.

To create an array of full-length oligonucleotides another approach may be used, involving a coding surface (FIG. 12B). Here oligonucleotides synthesized in each array feature have a different 5′ region. Capturing oligonucleotides complementary to these 5′ regions are synthesized on another array. Features are located in such a way that complementary sequences during replication are opposite to each other. Such approach is diffusion safe, because oligonucleotides diffused to a neighbor feature would not hybridize to the target surface.

### DESCRIPTION OF FIGURES

FIG. 1: Hybridization-based binding of nucleic acids to the target surface. (A) Hybridization directly to the oligonucleotides attached to the target surface. (B) Binding to the target surface by hybridization through adapter oligonucleotides.

FIG. 2: Replication of 2D distributed nucleic acid molecules to a oligonucleotide coated target surface.

FIG. 3: Examples of enzymatic reactions which may be performed with replicated nucleic acid molecules (hybridization probes) on the target surface.
(A) Ligation. (B) Primer extension.
Subsequently, replicated nucleic acid molecules can be sequenced on the target surface, using the oligonucleotides on the target surface to start sequencing-by-synthesis or replicated nucleic acid molecules may be amplified, for example by rolling circle amplification (RCA), in situ PCR or bridge PCR.

FIG. 4: Scheme of rolling circle amplification of the replicated nucleic acid molecules (hybridization probe). Replicated nucleic acid molecule is circularised and amplified using oligonucleotides on the target surface first as a template for ligation and then as a primer for amplification.

FIG. 5: Cy3-labeled oligonucleotides #003 were hybridized to the slides #1 a (with oligonucleotides #001 deposited to form figure “1”) and to the slide #2 (with oligonucleotides #002 deposited to form figure “2”).

FIG. 6: Transfer of Cy3-labeled oligonucleotide #003 hybridized to the slide #1b_hybr to the slide #3. The surface of the target slide #3 is covered with covalently immobilised oligonucleotides #001 (grey area), which is complementary to #003.

FIG. 7: Scheme of replication of oligonucleotides from DEAE nitrocellulose membrane.

FIG. 8: Scheme of replication of oligonucleotides distributed within the gel layer.

FIG. 9: Scheme of replication of oligonucleotides gradually releasing from the original surface.

FIG. 10: Scheme of replication of polyadenylated RNA from a tissue section. PolyA tail of mRNA hybridizes to the oligo(dT) immobilised on the target surface.

FIG. 11: Scheme of replication of gene-specific hybridization probe from paraformaldehyde fixed tissue sections after in situ hybridization. (A) Structure of the probe for in situ hybridization. 5′ part is gene-specific and hybridizes to the mRNA in the tissue section. 3′ region of the probe is complementary to the oligonucleotides on the target surface. (B) Replication of hybridized probes to the oligonucleotide coated target surface.

FIG. 12: Selective replication of full-length oligonucleotides by hybridization to the sequences complementary to 5′ ends of oligonucleotides. (A) All synthesized oligonucleotides have the same sequence on 5′ end. (B) Synthesized oligonucleotides in each area of the oligonucleotide array have a different sequence on 5′ end. The target surface is organized as an array of oligonucleotides complementary to 5′ ends of full-length oligos.

FIG. 13: Expression profiling and locus specific sequencing in tissue sections. (A) Structure of the probes for in situ hybridization. Internal part is gene-specific and hybridizes to the mRNA in the tissue section. 5′ and 3′ end regions of the probe are sequencing adapters and are complementary to the oligonucleotides on the target surface. (B) Structure of the probes for locus specific sequencing. After primer extension and ligation the internal part became a copy of a specific gene locus. 5′ and 3′ end regions of the ligated probe are sequencing adapters and are complementary to the oligonucleotides on the target surface. (C) Replication of hybridized probes on the target surface for sequencing.

FIG. 14: (A) Structure of hybridization probe suitable for (i) hybridization to transcripts in tissue section, (ii) hybridization to the SOLiD P1 region of sequencing templates and having ilium. region 1 necessary for Illumina SGS. (B) Structure if the SOLiD sequencing template attached to the bead. (C-F) Scheme of adding a code to hybridization probes using primer extension. Hybridization probes transferred from tissue section slide hybridize to the P2 region (C). Hybridization probes are extended (D). Internal sequence of the sequencing template which marks the position of the bead on the SOLiD flowcell is now added to the hybridization probe sequence, thus marking the position of the transfer nd of original transcript in tissue section. To introduce ilium. region 2 necessary for Illumina SGS, coded hybridization probes are PCR amplified. One of PCR primers has a P1-complementary 3′ end and ilium. region 2 5′ tail; another primer correspond to ilium. region 1 (E). Resulting double-stranded molecules are suitable for Illumina SGS.

FIG. 15: Position coding involving sequenced SOLiD flowcell as a coding surface. Hybridization probes are transferred to a coding surface covered with beads. Each bead is characterised by a specific coding sequence.

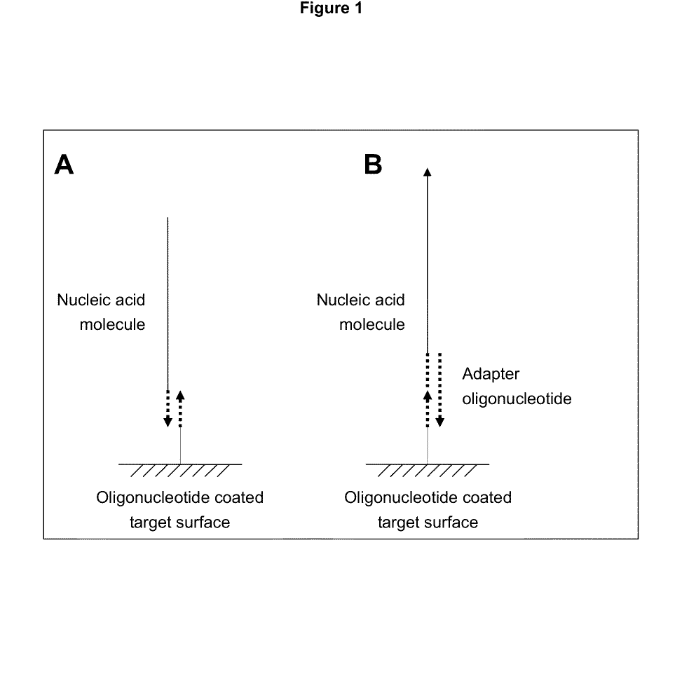

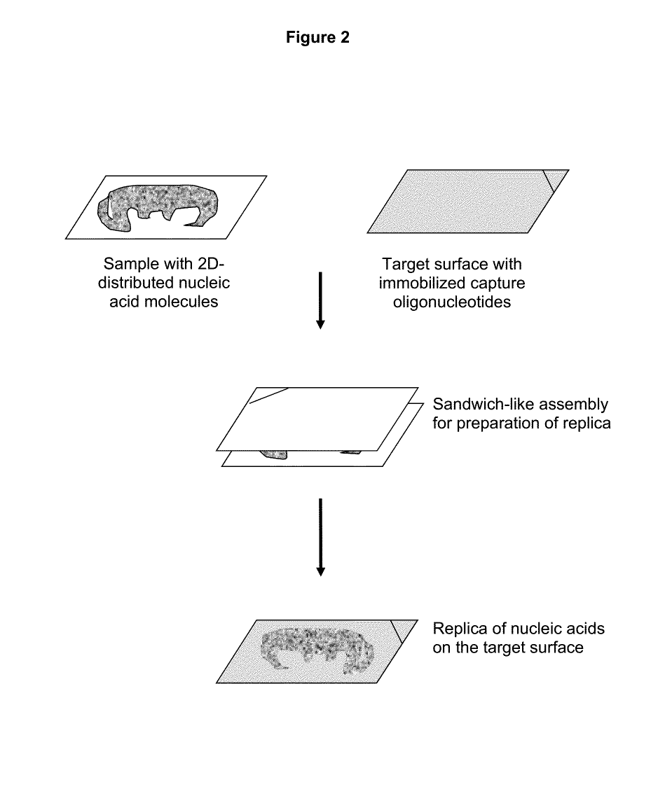

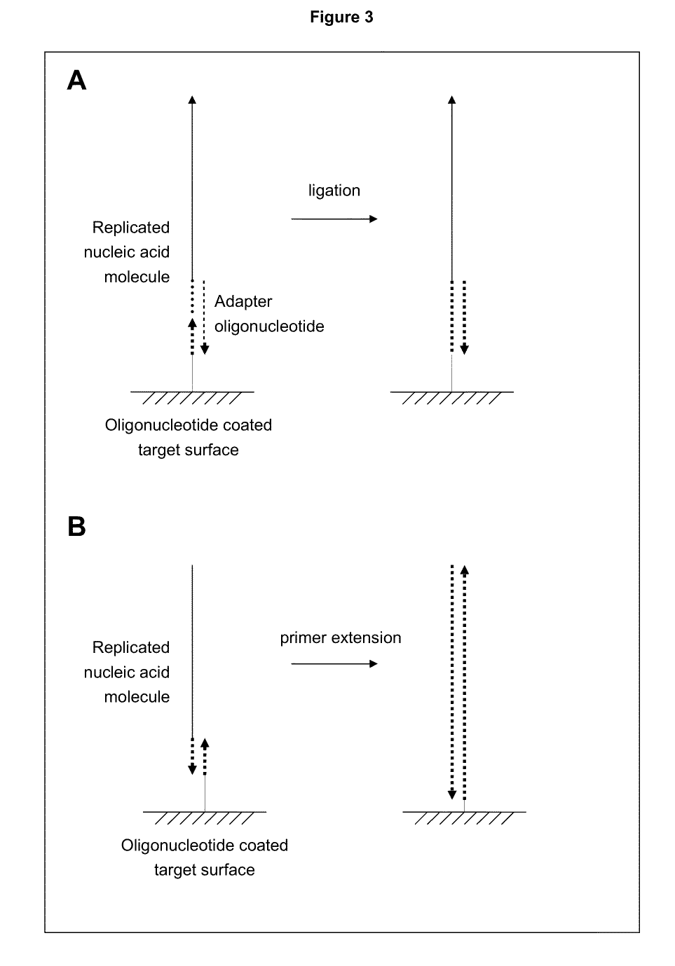

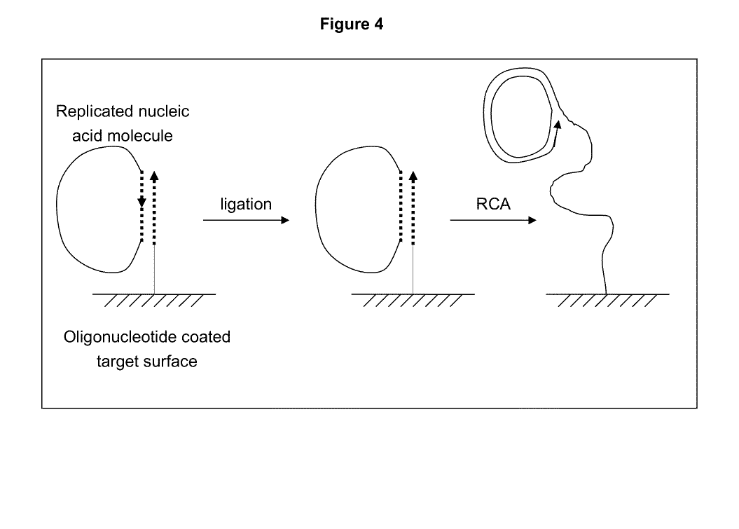

### EXAMPLES

### Example 1

### Replication of Oligonucleotides Attached to the Original Surface by Hybridization

### Consumables

Epoxy-modified glass slides: Nexterion Epoxysilane 2-D surface Slide E kit (Schott, #1066643)

Hybridization chambers: Secure Seal (Grace bio-labs, #SA500)

Oligonucleotides:
SH-modified oligonucleotides for immobilization on the epoxy slides:

#001
(SEQ ID NO: 1)
5′ SH-TTTTTTTTTTAATGATACGGCGACCACCGA 3′
#002
(SEQ ID NO: 2)
5′ SH-TTTTTTTTTTCAAGCAGAAGACGGCATACGA 3′

The unique sequences correspond to the sequences of oligonucleotides immobilized on the Illumina sequencing flowcells;

Cy3-labeled fluorescent hybridization probe:

#003
(SEQ ID NO: 3)
5′ Cy3-AGAGTGTAGATCTCGGTGGTCGCCGTATCATT 3′

Partly complementary to oligonucleotides #001, complementary sequence is underlined.

### Slides Prepared

SH-modified oligonucleotide #001 was immobilized on five epoxy slides: on three slides—in a recognizable pattern and on the other two—over the whole surface. SH-modified oligonucleotide #002 was immobilized on three epoxy slides: on one slide—in a recognizable pattern and on the other two—over the whole surface.

1. 40 μM SH-modified oligonucleotides were diluted to 20 μM by adding the equal volume of the 2× Nexterion Spot solution.

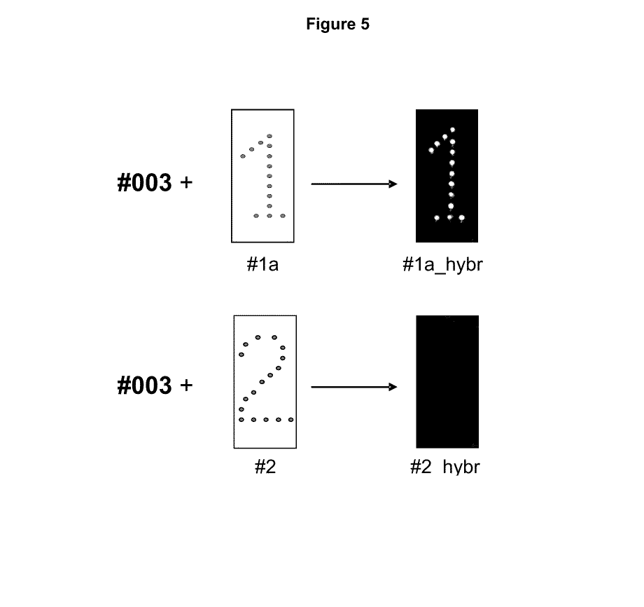

2. Oligonucleotide solutions were deposited on slides:
Slides #1a, #1b and #1c: 1 μl drops of oligonucleotide #001 were deposited to form a figure “1” (see FIG. 5, #1a):
Slide #2: 1 μl drops of oligonucleotide #002 were deposited to form a figure “2” (see FIG. 5, #2)
Slides #3&4: were laid upon each other with 3 mm wide and 0.2 mm thick spacers along the long sides and oligonucleotide #001 was pipetted to fill the space between the two slides;
Slides #5&6: were laid upon each other with 3 mm wide and 0.2 mm thick spacers along the long sides and oligonucleotide #002 was pipette to fill the space between the two slides.

Further all slides (#1a, b, c-6) were handled the same way.

3. Slides with deposited oligonucleotides were incubated in a humidity chamber at room temperature for 30 min to ensure quantitative immobilization.

4. Slides were washed at room temperature:
for 5 min in 0.1% Triton® X-100;
two times for 2 min in 1 mM HCl solution;
for 10 min in 100 mM KCl solution;
for 1 min in bidistilled water.

5. Blocking was performed:
incubating for 15 min in 1× Nexterion Blocking Solution at 50° C.;
rinsing for 1 min in bidistilled water at room temperature.

6. Slides with immobilized oligonucleotides were dried under nitrogen stream and stored in dry atmosphere in an excicator.

### Hybridization of Cy3-Labeled Oligonucleotides to the Slides #1a, b, c and #2.

1. Cy3-labeled oligonucleotides #003 solution was prepared: 10 nM oligonucleotide in 90% Nexterion Hybridization buffer.
2. Hybridization chambers were placed over the areas with spots on slides #1a, #1b, #1c and #2, the labelled oligonucleotides solution was added to the chamber.
3. Slides were incubated for 1 hour at 42° C. in the PCR machine with glass slides adapter.
4. Hybridization chambers were removed and slides were washed at room temperature:
for 10 min in (2×SSC, o.2% SDS);
for 10min in 2×SSC;
for 10min in 0.2×SSC.
   5. Slides #1b and #1c with hybridized Cy-3 labeled oligonucleotide (#1b_hybr and #1c_hybr) were left in 0.2×SSC at room temperature for ˜1 hour, till the transfer experiment was performed.
   6. Slides #1 a and #2 with hybridized Cy-3 labeled oligonucleotide (#1a_hybr and #2_hybr) were dried under nitrogen stream and scanned on the Affymetrix 428 Array Scanner.

On slide #1a_hybr a fluorescent pattern of the figure “1” was obtained (see FIG. 5). No fluorescent signal was observed on the slide #2_hybr.

Transfer of the Cy-Labeled Oligonucleotide #003 Hybridized to Slides #1b_hybr and #1c_hybr to the Slides #3 and #5

Cy3-labeled oligonucleotides #003 hybridized to the slide #1b_hybr was transferred to the slide #3 covered with oligonucleotide #001, complementary to #003. Cy-3 labeled oligonucleotide #003 hybridized to the slide #1c_hybr was transferred to the slide #5 covered with oligonucleotide #002, not complementary to #003.

1. ˜25 μl of Nexterion Hybridization buffer was pipetted on the oligonucleotide covered surfaces of slides #3 and #5, to which the Cy-3 labeled oligonucleotide had to be transferred.
2. Slides #1b_hybr and #1c_hybr with the hybridized fluorescent oligonucleotide #003 were placed over the drop of Nexterion Hybridization buffer on slides #3 and #5 respectively.
3. The sandwiches of slides #1b_hybr/#3 and #1c_hybr/#5 were placed in separate plastic bags.
4. The slides in both sandwiches were pressed tightly to each other with paper clips to let the hybridization buffer squeeze out into the bag.
5. Bags with slide sandwiches were placed in a beaker with boiling water for 3 min.
6. Bags were transferred to a beaker with 42° C. water for 15 min.
7. Bags were transferred to room temperature; sandwiches were taken out and disassembled. All slides were washed, blocked, dried out and scanned as described in the “Hybridization” section.

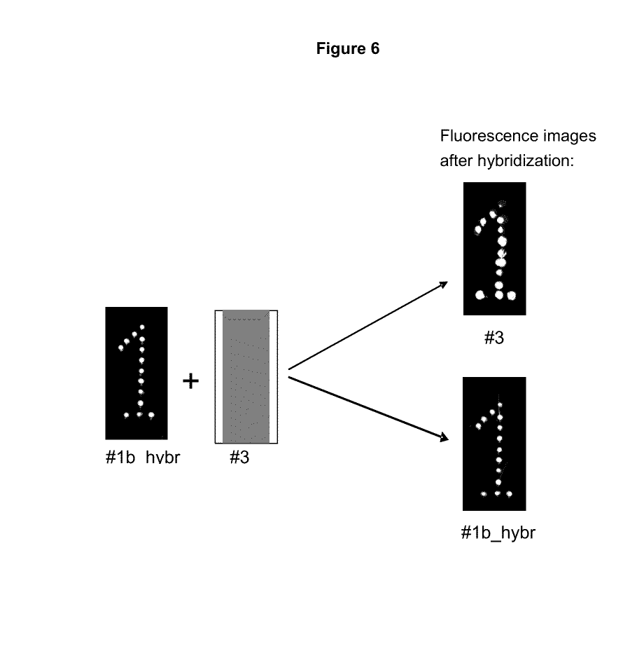

On slide #3a mirror replica of the fluorescent pattern of the figure “1” from slide #1b_hybr was obtained. Thus, the Cy-3 labeled oligonucleotide #003 hybridized to the slide #1b_hybr has been transferred to the surface of slide #3 (see FIG. 6). No transfer of oligonucleotide #003 from slide #1c_hybr to the slide #5 was observed.

### Example 2

### Replication of Oligonucleotides from DEAE Nitrocellulose Membrane to the Oligonucleotide-Coated Glass Slide

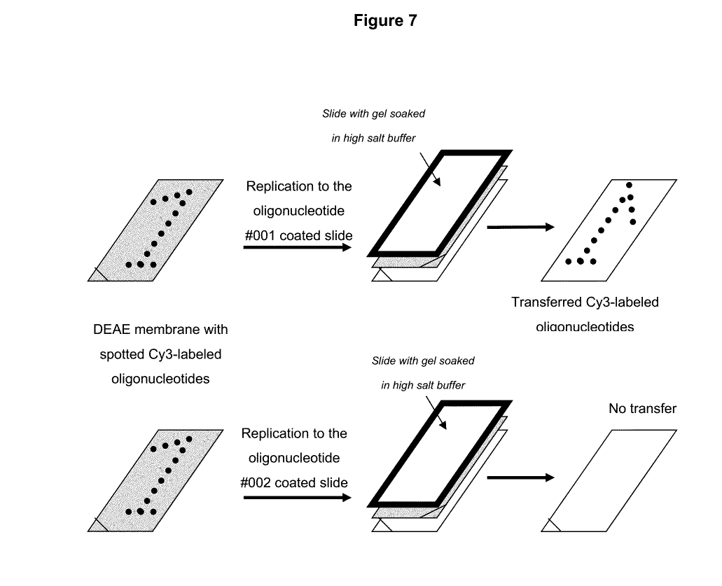

The scheme of the experiment is shown on FIG. 7. The experiment is based on the ability of DEAE membrane to bind DNA molecules in the low salt buffer and to release them in the high salt buffer.

Nexterion glass slides were coated over the whole surface with #001 and #002 oligonucleotides as described in the Example 1.

#001
5′ SH-TTTTTTTTTTAATGATACGGCGACCACCGA 3′
#002
5′ SH-TTTTTTTTTTCAAGCAGAAGACGGCATACGA 3′

Standard glass slides were treated with Bind-silane and covered with 4 mm thick 12% polyacrylamide gel. The gel was impregnated with High Salt Buffer (50 mM TrisCl pH 7.0, 10 mM EDTA, 1M NaCl) and stored in a fridge in plastic bags to prevent drying of the gel. Gel was used to provide a uniform delivery of high salt buffer over the whole area of the DEAE membrane.

DEAE cellulose membranes (DE 81 DEAE chromatography paper, Whatman) of the same size as glass slides were soaked for 5 min in 10 mM EDTA (pH 8.0), 5 min in 0.5N NaOH and finally washed thoroughly in TE, pH 7.5. 20 μM Cy3-labeled fluorescent oligonucleotide #003 was deposited on the DEAE membranes in 0.15 μl TE buffer (10 mM TrisCl pH 7.5, 1 mM EDTA) drops to form a figure “1” (FIG. 7). When the #003 drops soaked into the DEAE membranes, membranes were washed thoroughly in Low Salt Buffer (50 mM TrisCl pH 7.0, 10 mM EDTA, 0.1M NaCl). Then they were placed over the #001 and #002 oligonucleotide coated slides. The sandwich-like assemblies were placed on the 42° C. in the PCR machine with glass slides adapter (with glass slides below) and the slides covered with gel were placed over the DEAE membrane. The sandwiches were left at 42° C. for 15 minutes.

Sandwiches were cooled to 0° C. and disassembled. #001 and #002 oligonucleotide coated glass slides were washed and scanned on the Affymetrix 428 Array Scanner to analyse the distribution of the Cy3 fluorescent signal as described in the Example 1. As a result fluorescent image in form of a mirror-reflected figure “1” appeared on #001 coated, but not on the #002 coated glass slide.

### Example 3

### Replication of Oligonucleotides from the Gel Layer to the Oligonucleotide-Coated 3D-Epoxy Hydrophilic Sinter Membrane

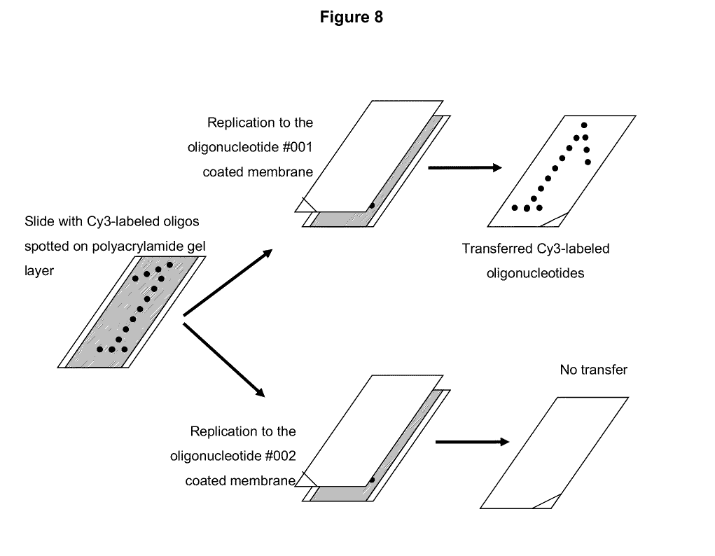

The scheme of the experiment is shown on FIG. 8.

3D-Epoxy hydrophilic sinter membranes (Polyolefin sinter, PolyAn GmbH, Berlin) were coated over the whole surface with #001 and #002 oligonucleotides using the same procedure as described in the Example 1.

#001
5′ SH-TTTTTTTTTTAATGATACGGCGACCACCGA 3′
#002
5′ SH-TTTTTTTTTTCAAGCAGAAGACGGCATACGA 3′

The glass slides were treated with Bind-silane and covered with 0.2 mm thick 12% polyacrylamide gel. The gel was impregnated with Nexterion Hybridization buffer and cooled to 0° C. 20 μM Cy3-labeled fluorescent oligonucleotide #003 was deposited on the gel in 0.15 μl Nexterion Hybridization buffer drops to form a figure “1” (FIG. 8). When the #003 oligonucleotide drops soaked into the gel, #001 and #002 oligonucleotide coated sinter membranes were placed on the gel coated slides at 0° C. The sandwich-like assemblies were sealed in plastic bags and incubated at 42° C. in a humidity chamber for 15 minutes.

Sandwiches were again cooled to 0° C. and disassembled. #001 and #002 oligonucleotide coated 3D-Epoxy hydrophilic sinter membranes were washed and scanned on the Affymetrix 428 Array Scanner as described in the Example 1 to analyse the distribution of the Cy3 fluorescent signal. As a result fluorescent image in form of a figure “1” appeared on #001 coated, but not on the #002 coated sinter membrane.

### Example 4

### Replication of Oligonucleotides Gradually Releasing from the Original Surface

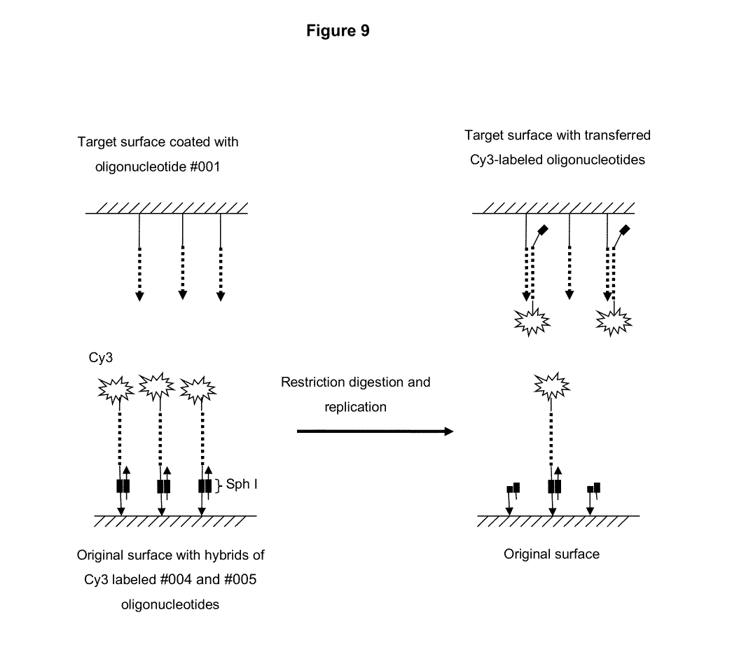

The scheme of experiment 3 is shown in FIG. 9.

Glass slides coated with #001, #002 and with Cy3-labeled #004 fluorescent oligonucleotides were prepared as described in Example 1. Oligonucleotides #001 and #002 were immobilized over the whole surface. The 3′ SH modified Cy3-labeled #004 fluorescent oligonucleotide was deposited to form a figure “1”.

#001
(SEQ ID NO: 1)
5′ SH-TTTTTTTTTTAATGATACGGCGACCACCGA 3′
#002
(SEQ ID NO: 2)
5′ SH-TTTTTTTTTTCAAGCAGAAGACGGCATACGA 3′
#004
(SEQ ID NO: 4)
5′ Cy3-AGAGTGTAGATCTCGGTGGTCGCCGTATCATTC
AGCATGCACTTTTTTTTTT-SH 3′

#004 is partly complementary to oligonucleotide #001 (underlined sequence) and contains a SphI recognition site (sequence is marked bold).

Short oligonucleotide #005 complementary to #004 was hybridized to slides with oligonucleotide #004 pattern to form double-stranded Sph I restriction site.

#0015
(SEQ ID NO: 5)
5′ AAAGTGCATGCTGAAT 3′

Slides were cooled down to 0° C. and washed with 1× NEBuffer 3.1 restriction buffer. Ice-cold 1× NEBuffer 3.1 with small amount of Sph I restriction endonuclease was placed on #001 and #002 oligonucleotide coated slides and these slides were covered with #005/#004 coated slides. The sandwich-like assemblies were incubated at 42° C. in a humidity chamber for 1 hour.

Sandwiches were cooled to 0° C. and disassembled. #001 and #002 oligonucleotide coated slides were washed and scanned on the Affymetrix 428 Array Scanner as described in Example 1 to analyse the distribution of the Cy3 fluorescent signal. As a result fluorescent image in form of a figure “1” appeared on #001 coated, but not on the #002 coated glass slide.

### Example 5

### Replication of Polyadenylated RNA from Frozen Tissue Section

Highly parallel molecular biology methods, for example, sequencing, cannot be performed directly in tissue sections. It would be advantageous to be able to transfer molecules from the tissue section to another surface, where appropriate methods of analysis could be performed. This example shows how mRNA may be replicated from tissue sections to another surface.

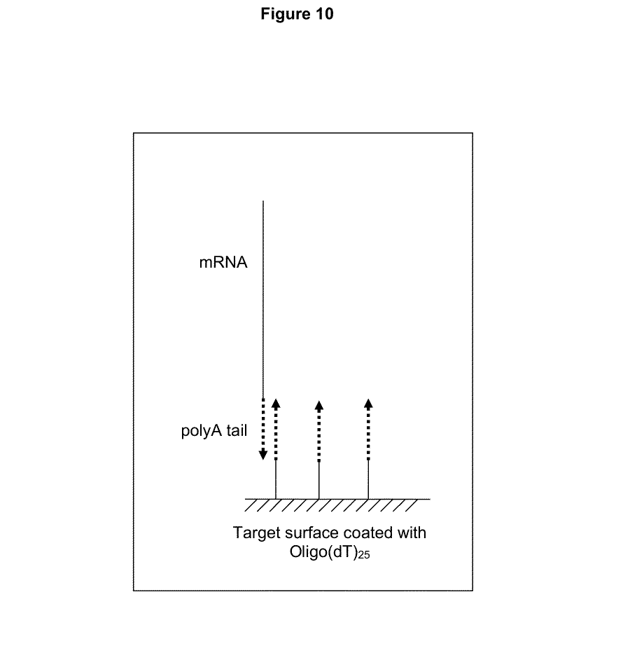

The scheme of experiment is shown on FIG. 10.

Oligo(dT)₂₅ and Oligo(dA)₂₅ coated glass slides were prepared as described in Example 1. Oligo(dA)₂₅ coated glass slides were used as a control for non-specific binding.

Oligo(dT)₂₅
(SEQ ID NO: 6)
5′ SH-TTTTTTTTTTTTTTTTTTTTTTTTT 3′
Oligo(dA)₂₅
(SEQ ID NO: 7)
5′ SH-AAAAAAAAAAAAAAAAAAAAAAAAA 3′

10 μm thick 14 days mouse embryo cryosections were placed on the Oligo(dT)₂₅ and Oligo(dA)₂₅ coated slides. Slides were cooled to 0° C. Ice-cold 1 mm thick 12% polyacrylamide gel attached to the glass slide and impregnated with Lysis/Binding Buffer (Dynabeads® mRNA DIRECT™ Kit, Ambion #61011) were put on the slides with tissue sections, such that the gel covered the cryosection. Sandwiches were incubated at room temperature for 25 minutes.

Sandwiches were cooled to 0° C. and disassembled. Oligonucleotide-coated slides were washed with Washing Buffer A and then with Washing Buffer B (Dynabeads® mRNA DIRECT™ Kit, Ambion #61011). After additional washing with 1× Reverse transcription buffer (SuperScript® III First-Strand Synthesis System, Invitrogen #18080-051), the first strand synthesis reaction with Digoxigenin Labeled dUTP (Roche, #11573179910) was performed under the cover glass. The slide was washed with 5×SSC buffer and stained with BCIP/NBT Assay Kit.

As a result Indigo-colored picture resembling the form of the cryosection appeared on Oligo(dT)₂₅ coated, but not on the Oligo(dA)₂₅ coated glass slide.

As an additional control Oligo(dT)₂₅ and Oligo(dA)₂₅ oligonucleotide coated glass slides after disassembling and washing with Washing Buffers A and B were incubated 5 min at 70° C. with 10 mM Tris-HCl (Elution buffer, Dynabeads® mRNA DIRECT™ Kit) to elute attached mRNA. RT-PCR with primers for mouse G3PDH gene showed that mRNA was transferred to Oligo(dT)₂₅ coated, but not on the Oligo(dA)₂₅ coated glass slide.

### Example 6

### Replication of Gene-Specific Hybridization Probe After In Situ Hybridization with Paraformaldehyde Fixed Tissue Section

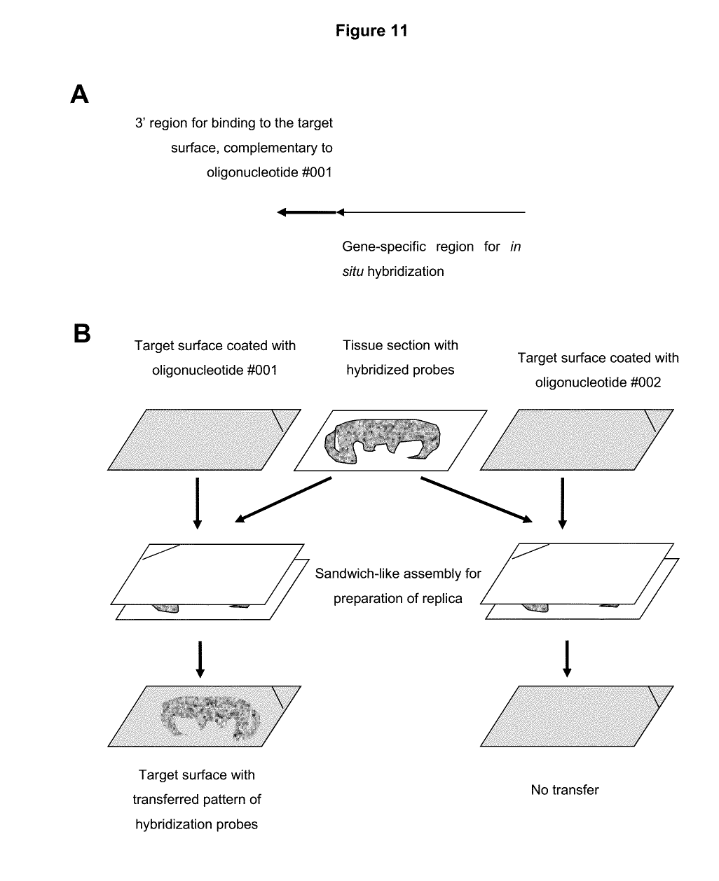

The scheme of experiment is shown in FIG. 11.

Glass slides coated with #001 and #002 oligonucleotides were prepared as described in Example 1.

#001
5′ SH-TTTTTTTTTTAATGATACGGCGACCACCGA 3′
#002
5′ SH-TTTTTTTTTTCAAGCAGAAGACGGCATACGA 3′

Frozen sections were fixed in 4% paraformaldehyde. In situ hybridization was performed with DIG labeled single stranded RNA probes for the G3PDH gene (sequences of three RNA probes used for hybridization are shown below). The probes contained 5′ G3PDH-specific area and 3′ region complementary to the #001 oligonucleotide (underlined sequence).

(SEQ ID NO: 8)
5′ TGTGAGGGAGATGCTCAGTGTTGGGGGCCGAGTTGGGATAGGGCCTCTCTTGCTCAGTGTCCTTGC
TGGGGTGGGTGGTCCAGGGTTTCTTACTCCTTGGAGGCCATGTAGGCCATGAGGTCCACCACCCTGTT
GCTGTAGCCGTATTCATTGTCATACCAGGAAATGAGCTTGACAAAGTTGTCATTGAGAGCAATGCCAG
CCCCGGCATCGAAGGTGGAAGAGTGGGAGTTGCTGTTGAAGTCGCAGGAGACAACCTGGTCCTCAGTG
TAGCCCAAGATGCCCTTCAGTGGGCCCTCAGATGCCTGCTTCACCACCTTTCGGTGGTCGCCGTATCA
TT 3′
(SEQ ID NO: 9)
5′ CTTGATGTCATCATACTTGGCAGGTTTCTCCAGGCGGCACGTCAGATCCACGACGGACACATTGGG
GGTAGGAACACGGAAGGCCATGCCAGTGAGCTTCCCGTTCAGCTCTGGGATGACCTTGCCCACAGCCT
TGGCAGCACCAGTGGATGCAGGGATGATGTTCTGGGCAGCCCCACGGCCATCACGCCACAGCTTTCCA
GAGGGGCCATCCACAGTCTTCTGGGTGGCAGTGATGGCATGGACTGTGGTCATGAGCCCTTCCACAAT
GCCAAAGTTGTCATGGATGACCTTGGCCAGGGGGGCTAAGCAGTTGGTGGTCGGTGGTCGCCGTATCA
TT 3′
(SEQ ID NO: 10)
5′ TGCAGGATGCATTGCTGACAATCTTGAGTGAGTTGTCATATTTCTCGTGGTTCACACCCATCACAA
ACATGGGGGCATCGGCAGAAGGGGCGGAGATGATGACCCTTTTGGCTCCACCCTTCAAGTGGGCCCCG
GCCTTCTCCATGGTGGTGAAGACACCAGTAGACTCCACGACATACTCAGCACCGGCCTCACCCCATTT
GATGTTAGTGGGGTCTCGCTCCTGGAAGATGGTGATGGGCTTCCCGTTGATGACAAGCTTCCCATTCT
CGGCCTTGACTGTGCCGTTGAATTTGCCGTGAGTGGAGTCATACTGGAACATGTATCGGTGGTCGCCG
TATCATT 3′

After hybridization and washing with Nexterion Hybridization buffer the slides with tissue sections were assembled with #001 and #002 coated slides. Replication and disassembling of a sandwich was performed as described in Example 1. Oligonucleotide-coated slides were washed with 5×SSC buffer and stained with BCIP/NBT Assay Kit.

As a result Indigo-colored picture resembling the form of embryo section appeared on #001 coated, but not on the #002 coated glass slide.

### Example 7

### Using Hybridization-Based Selective Replication for Purification of Full-Length Oligonucleotides

Hybridization-based replication method is highly selective. Only those nucleic acid molecules will be replicated, which have a sequence complementary to the capturing oligonucleotides.

Selectivity may be used for purification of full-length oligonucleotides after on-surface synthesis. Chemical synthesis of oligonucleotides is performed from 3′ to 5′ end. For selection of full-length oligonucleotides it is possible to use capturing oligonucleotides complementary to 5′ regions of synthesized oligonucleotides.

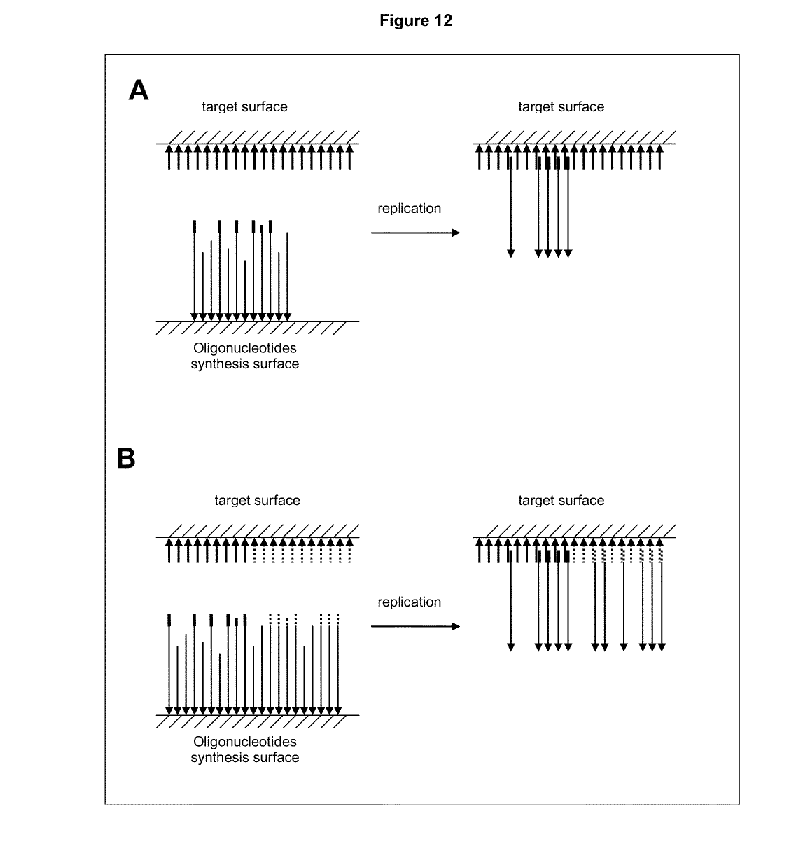

One scheme of purification of full-length oligonucleotides is shown in FIG. 12A. The synthesized oligonucleotides should have the same 5′ regions complementary to the capturing oligonucleotides. During replication of synthesized oligonucleotides to the target surface, only full length oligonucleotides get captured.

The scheme for purification of full-length oligonucleotides without constant 5′ regions is shown in FIG. 12B. In this case a special target surface covered with different types of oligonucleotides should be prepared. Specific oligonucleotides should be located in such a way that complementary sequences are opposite to each other during replication. A target surface covered with short capturing oligonucleotides will be used for purification of full-length long oligonucleotides. The approach is diffusion safe, because oligonucleotides diffused to neighbor areas would not hybridize to the target surface.

### Example 8

### Expression Profiling in Tissue Sections

Example 6 demonstrates how in situ hybridized probes may be transferred from a tissue section to another surface. In principle, any number of different probes may be transferred in parallel in such a way. But if transferred molecules would be stained as in Example 6, it would not be possible to recognize positions of individual genes in common picture. However, if transferred molecules would be sequenced on the target surface; then distribution of any number of genes may be correctly determined in parallel. The only restriction is not to overload the target surface above the maximum density of molecules for the particular sequencing method.

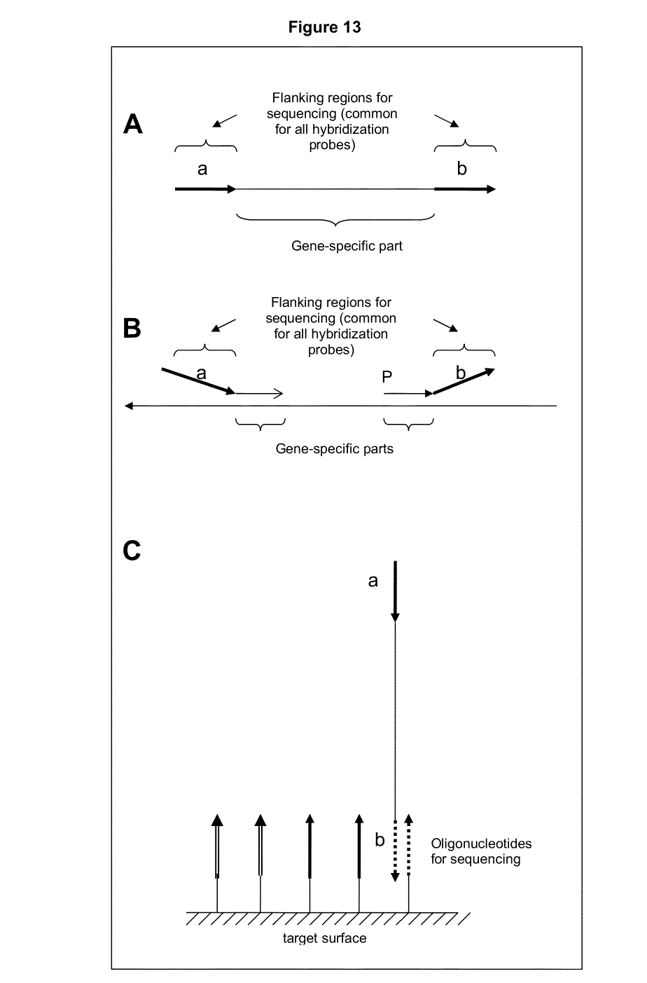

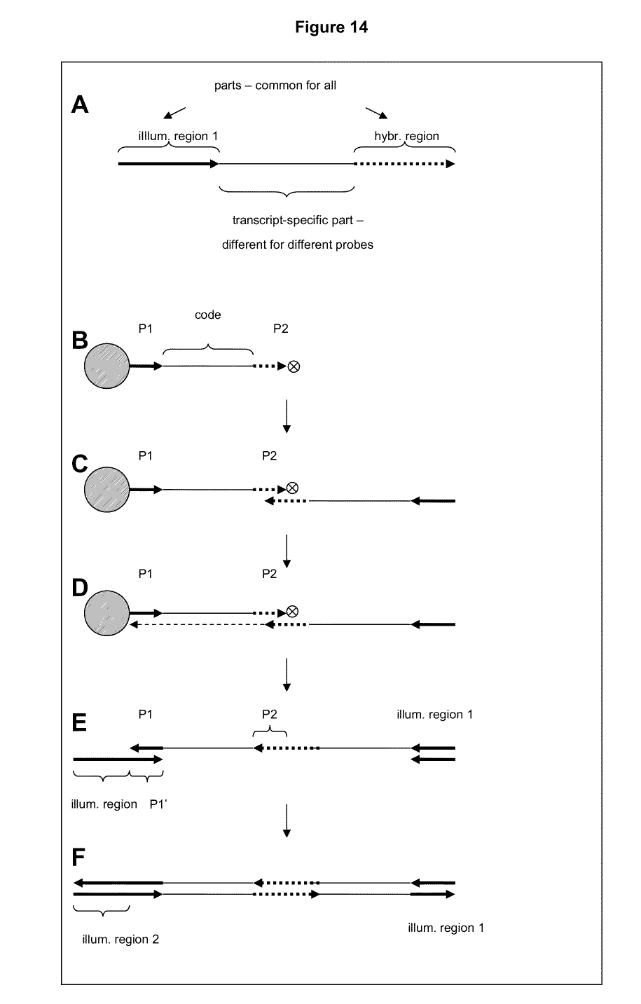

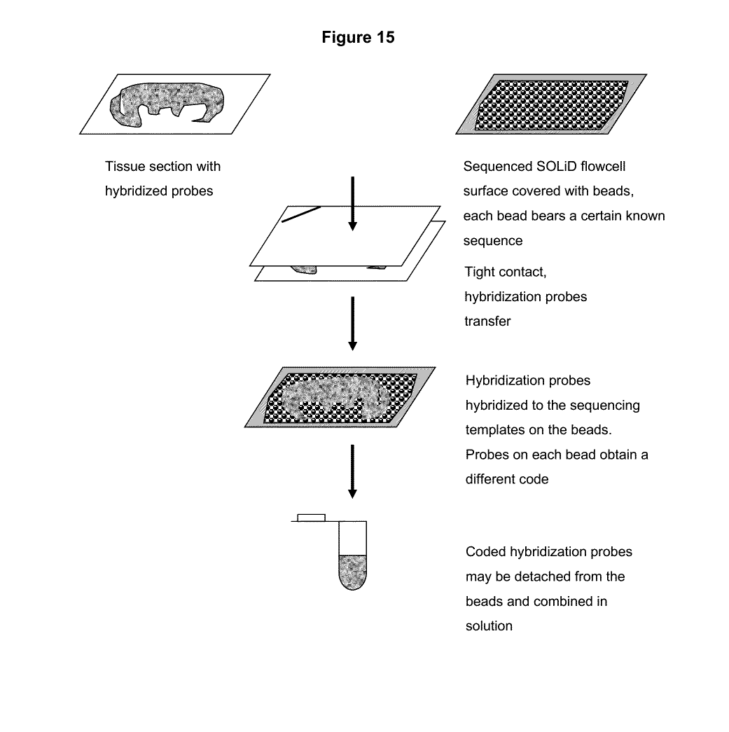

The scheme of positional expression profiling is shown in FIG. 13.

After fixation of frozen sections with paraformaldehyde, in situ hybridization with a set of gene-specific probes should be performed. The structure of gene-specific probes is shown in FIG. 13A. The internal part of probes is gene-specific. It is used for hybridization with target mRNA and for recognition of probes in sequencing reaction. 5′ and 3′ end regions (only 3′ end region for third-generation single-molecule sequencing) are for the sequencing (depending on the platform: for bridge amplification, primer extension, etc.)

After in situ hybridization non-hybridized probes should be washed off. Specifically bounded probes should be replicated on a surface for sequencing (FIG. 13B). For each specific gene (i) the distribution of correspondent probes on the target surface resembles a distribution of gene-specific mRNA in tissue section; (ii) the amount of sequenced probes is proportional to the expression level of the gene.

### Example 9

### Locus Specific Sequencing in Tissue Sections

The method for expression profiling in tissue sections described in Example 8 may be adopted for the analysis of DNA within a tissue section. The only difference is that locus-specific probes should be hybridized with DNA and designed to take copy of a sequence of a particular genomic locus (such as probes for GoldenGate technology/Illumina/, FIG. 13B).

1-20. (canceled)

21. Method of transfer of nucleic acid molecules to a target surface, preserving their relative spatial distribution resembling the original distribution, wherein said nucleic acids molecules are fixed on said target surface by hybridization, comprising the following steps:
a) providing the sample containing nucleic acid molecules located either on the surface of the sample or within the sample;
b) providing a target surface with immobilized oligonucleotides;
c) if the nucleic acid molecules are not attached to the sample, providing conditions to minimize shift of the nucleic acid molecules from the original positions on or within the sample; or
c) if nucleic acid molecules are attached to the sample, providing conditions for releasing the nucleic acid molecules;
d) assembling the sample and the target surface in such a way, that a distance from positions of said nucleic acids to the target surface is smaller than the distortion acceptable for the replica and with a medium in between sample and target surface;
e) providing conditions for diffusion of the nucleic acid molecules from the sample to the target surface and hybridization-based binding of nucleic acid molecules to the oligonucleotides on the target surface.

22. Method according to claim 21, wherein step c′) (releasing of nucleic acid molecules) is performed after step d) (assembling of sample and target surface).

23. Method according to claims 21, comprising after step e) further step f) providing conditions for slowing down the formation of new hybrids of nucleic acid molecules and oligonucleotides.

24. Method according to claim 21, comprising the following steps if the nucleic acid molecules are not attached to the sample:
a) providing the sample containing nucleic acid molecules located either on the surface or within the sample;
b) providing a target surface with immobilized oligonucleotides;
c) providing conditions to minimize shift of the nucleic acid molecules from the original positions on or within the sample;
d) assembling the sample and the target surface with a medium in between sample and target surface;
e) providing conditions for diffusion of the nucleic acid molecules from the sample to the target surface and hybridization-based binding of the nucleic acid molecules to the oligonucleotides on the target surface;
or if the nucleic acid molecules are attached to the sample
a) providing the sample containing nucleic acid molecules located either on the surface or within the sample;
b) providing a target surface with immobilized oligonucleotides;
c) providing conditions for releasing the nucleic acid molecules;
d) assembling the sample and the target surface with a medium in between sample and target surface;
e) providing conditions for diffusion of the nucleic acid molecules from the sample to the target surface and hybridization-based binding of the nucleic acid molecules to the oligonucleotides on the target surface;
or
a) providing the sample containing nucleic acid molecules located either on the surface or within the sample;
b) providing a target surface with immobilized oligonucleotides;
d) assembling the sample and the target surface with a medium in between sample and target surface;
c) providing conditions for releasing the nucleic acid molecules;
e) providing conditions for diffusion of the nucleic acid molecules from the sample to the target surface and hybridization-based binding of the nucleic acid molecules to the oligonucleotides on the target surface.

25. Method according to claim 21, wherein said nucleic acid molecules located on the surface are located on a nucleic acid array or protein array, or wherein said nucleic acid molecules distributed within the sample are distributed in a gel layer, in tissue section, in cell or tissue array or in a block of tissue.

26. Method according to claim 21, wherein the target surface is a surface of a glass, plastic, metal, paper, or porous membrane target, optionally covered with gel, dendrimers or microbeads and wherein the oligonucleotides on said target surface are made of DNA, RNA, LNA, PNA or mixture or hybrids of those and immobilized on the target surface by covalent or non-covalent binding directly to the surface or through gel, dendrimers or other chemical compounds attached to the surface.

27. Method according to claim 21, wherein in step e) said hybridization-based binding occurs through adapter oligonucleotides which are complementary both to the nucleic acid molecules from the sample and to the immobilized oligonucleotides on the target surface.

28. Method according to claim 21, wherein assembling the sample and target surface in step d) is performed with a temperature low enough to slow down a shift of the nucleic acid molecules from the original positions on or within the sample.

29. Method according to claim 21, wherein nucleic acid molecules in the sample are held on the original positions by chemical- or enzyme-sensitive binding and said conditions for releasing of the nucleic acid molecules in step c′) are provided by a cleavage agent which destroys said binding and acts slow enough to ignore those molecules which change the positions before assembling sample against the target surface in step d).

30. Method according to claim 29, wherein the releasing of nucleic acid molecules by the cleavage agent is slowed down by decreasing concentration of said agent or by providing reaction conditions suppressing the activity of said agent at least partially.

31. Method according to claim 21, wherein the condition for releasing the nucleic acid molecules from the original positions in the sample in step c′ comprises increasing the temperature.

32. Method according to claim 31, wherein the nucleic acid molecules are held on the original positions in the sample by temperature-sensitive binding or the medium comprises a thermoactivated cleavage agent.

33. Method according to claim 32, wherein said temperature sensitive binding is done by hybridization or through thermolabile covalent bonds, abasic site or formaldehyde linkages, or wherein the thermoactivated cleavage agent is an enzyme.

34. Method according to claim 21, wherein the condition for releasing the nucleic acid molecules from the original positions in the sample in step c′) comprises changing the medium between the sample and the target surface.

35. Method according to claim 34, wherein the nucleic acid molecules are held on the original positions in the sample by hybridization and the new medium destabilizes hybridization by changing pH or ionic strength of the medium or by decreasing the melting temperature of the hybrid like formamide, or the nucleic acid molecules are held on the original positions in the sample by chemical- or enzyme-sensitive binding and said new medium contains a cleavage agent, and wherein either the sample or the target surface or both are permeable for the medium and during changing of the medium the assembly remains intact.

36. Method according to claim 21, wherein the condition for releasing the nucleic acid molecules from the original positions in the sample in step c′) comprises light and wherein the nucleic acid molecules are held on the original positions in the sample by photocleavable binding and wherein either the sample or the target surface are transparent for the light with required wavelength.

37. Method according to claim 21, wherein the sample, the target surface or both are subdivided into isolated regions, wherein the nucleic acid molecules can't cross the borders of said regions and wherein the regions are created by using a mask with isolated holes or by scratching the sample or the target surface.

38. Method according to claim 21, wherein the conditions for diffusion of nucleic acid molecules from the sample to the target surface are facilitated by liquid flow (blotting) or by electric field (electrophoresis).

39. Method according to claim 23, wherein the conditions for slowing down the formation of new hybrids of the nucleic acid molecules on the target surface comprises decreasing of the temperature of the sample or the target surface.

40. Use of replicas prepared according to claim 21 for sequencing of the nucleic acid molecules transferred from the sample, wherein said sequencing is performed directly on the target surface and the relative positions of the sequenced nucleic acid molecules resemble spatial distribution of the nucleic acid molecules in the sample.
